SEQUENCE LISTING (38 sequences)

SEQ ID NO: 1 - DNA, 1352 bp, Homo sapiens
tctatccaggatgtaaatgagcacactgctggcccatgcgcctcggggctgtagagggcagcctcagaggcactgggcattcctggcaccatggatgacgctgctgtcctcaagcgacgaggctacctcctggggataaatttaggagagggctcctatgcaaaagtaaaatctgcttactctgagcgcctgaagttcaatgtggcgatcaagatcatcgaccgcaagaaggcccccgcagacttcttggagaaattccttccccgggaaattgagattctggccatgttaaaccactgctccatcattaagacctacgagatctttgagacatcacatggcaaggtctacatcgtcatggagctcgcggtccagggcgacctcctcgagttaatcaaaacccggggagccctgcatgaggacgaagctcgcaagaagttccaccagctttccttggccatcaagtactgccacgacctggacgtcgtccaccgggacctcaagtgtgacaaccttctccttgacaaggacttcaacatcaagctgtccgacttcagcttctccaagcgctgcctgcgggatgacagtggtcgaatggcattaagcaagaccttctgtgggtcaccagcgtatgcggccccagaggtgctgcagggcattccctaccagcccaaggtgtacgacatctggagcctaggcgtgatcctctacatcatggtctgcggctccatgccctacgacgactccaacatcaagaagatgctgcgtatccagaaggagcaccgcgtcaacttcccacgctccaagcacctgacaggcgagtgcaaggacctcatctaccacatgctgcagcccgacgtcaaccggcggctccacatcgacgagatcctcagccactgctggatgcagcccaaggcacggggatctccctctgtggccatcaacaaggagggggagagttcccggggaactgaacccttgtggacccccgaacctggctctgacaagaagtctgccaccaagctggagcctgagggagaggcacagccccaggcacagcctgagacaaaacccgaggggacagcaatgcaaatgtccaggcagtcggagatcctgggtttccccagcaagccgtcgactatggagacagaggaagggcccccccaacagcctccagagacgcgggcccagtgagcttcttgcggcccagggaatgagatggagctcacggcttaaagcccaagctctgaagaagtcaagggtggagccagagaaggaaggcagtcccagatgagcctctattttcatcagcttcttctctctccccttgaacttggtaacccacatggttc

SEQ ID NO: 2 - DNA, 1254 bp, Homo sapiens
ttgaggacaacgcctgctggcccacatgacggggggatgtagacggcagcggcgccagtcgctcctggcaccatggacgatgccacagtcctaaggaagaagggttacatcgtaggcatcaatcttggcaagggttcctacgcaaaagtcaaatctgcctactctgagcgcctcaagttcaatgtggctgtcaagatcatcgaccgcaagaaaacacctactgactttgtggagagattccttcctcgggagatggacatcctggcaactgtcaaccacggctccatcatcaagacttacgagatctttgagacctctgacggacggatctacatcatcatggagcttggcgtccagggcgacctcctcgagttcatcaagtgccagggagccctgcatgaggacgtggcacgcaagatgttccgacagctctcctccgccgtcaagtactgccacgacctggacatcgtccaccgggacctcaagtgcgagaaccttctcctcgacaaggacttcaacatcaagctgtctgactttggcttctccaagcgctgcctgcgggacagcaatgggcgcatcatcctcagcaagaccttctgcgggtcggcagcatatgcagcccccgaggtgctgcagagcatcccctaccagcccaaggtgtatgacatctggagcctgggcgtgatcctgtacatcatggtctgcggctccatgccctatgacgactccgacatcaggaagatgctgcgtatccagaaggagcaccgtgtggacttcccgcgctccaagaacctgacctgcgagtgcaaggacctcatctaccgcatgctgcagcccgacgtcagccagcggctccacatcgatgagatcctcagccactcgtggctgcagccccccaagcccaagccaacgtcttctgcttccttcaagagggagggggagggcaagtaccgcgctgagtgcaaactggacaccaagacaggcttgaggcccgaccaccggcccgaccacaagcttggagccaaaacccagcaccggctgctggtggtgcccgagaacgagaacaggatggaggacaggctggccgagacctccagggccaaagaccatcacatctccggagctgaggtggggaaagcaagcacctagcatgacaatggccccgttgtgtgtggtgggggtcggggttggggggcatggtgcagtcggccttcacgtaaactaagtaggcaggtaggatctgaagaaggcaca

SEQ ID NO: 3 - DNA, 1058 bp, Homo sapiens
catcctaatacgactcactatagggctcgagcggcgcccgggcaggtgagaatgttctaacgctgggggcggctgcggatgaagtccttggggagaaaaggagcaggccaagggcgatggtggagtagagctgcctctcagaggcagcatgagctgagagggtgataggaaggcggcgctagacagcatggaggactttctgctctccaatgggtaccagctgggcaagaccattggggaagggacctactcaaaagtcaaagaagcattttccaaaaaacaccaaagaaaagtggcaattaaagttatagacaagatgggagggccagaagagtttatccagagattcctccctcgggagctccaaatcgtccgtaccctggaccacaagaacatcatccaggtgtatgagatgctggagtctgccgacgggaaaatctgcctggtgatggagctcgctgagggaggggatgtctttgactgcgtgctgaatggggggccactgcctgaaagccgggccaaggccctcttccgtcagatggttgaggccatccgctactgccatggctgtggtgtggcccaccgggacctcaaatgtgagaacgccttgttgcagggcttcaacctgaagctgactgactttggctttgccaaggtgttgcccaagtcacaccgggagctgagccagaccttctgcggcagtacagcctatgctgcccccgaggtgctgcagggcattccccacgatagcaaaaaaggtgatgtctggagcatgggtgtggtcctgtatgtcatgctctgtgccagcctaccttttgacgacacagacatccccaagatgctgtggcagcagcagaagggggtgtccttccccactcatctgagcatctcggccgattgccaggacctgctcaagaggctcctggaacccgatatgatcctccggccttcaattgaagaagttagttggcatccatggctagcaagcacttgataaaagcaatggcaagtgctctccaataaagtagggggagaaagcaaagcaaacccaaaaaaaa

SEQ ID NO: 4 - protein, 367 aa, Homo sapiens
MDDAAVLKRRGYLLGINLGEGSYAKVKSAYSERLKFNVAIKIIDRKKAPADFLEKFLPREIEILAMLNHCSIIKTYEIFETSHGKVYIVMELAVQGDLLELIKTRGALHEDEARKKFHQLSLAIKYCHDLDVVHRDLKCDNLLLDKDFNIKLSDFSFSKRCLRDDSGRMALSKTFCGSPAYAAPEVLQGIPYQPKVYDIWSLGVILYIMVCGSMPYDDSNIKKMLRIQKEHRVNFPRSKHLTGECKDLIYHMLQPDVNRRLHIDEILSHCWMQPKARGSPSVAINKEGESSRGTEPLWTPEPGSDKKSATKLEPEGEAQPQAQPETKPEGTAMQMSRQSEILGFPSKPSTMETEEGPPQQPPETRAQ

SEQ ID NO: 5 - protein, 358 aa, Homo sapiens
MDDATVLRKKGYIVGINLGKGSYAKVKSAYSERLKFNVAVKIIDRKKTPTDFVERFLPREMDILATVNHGSIIKTYEIFETSDGRIYIIMELGVQGDLLEFIKCQGALHEDVARKMFRQLSSAVKYCHDLDIVHRDLKCENLLLDKDFNIKLSDFGFSKRCLRDSNGRIILSKTFCGSAAYAAPEVLQSIPYQPKVYDIWSLGVILYIMVCGSMPYDDSDIRKMLRIQKEHRVDFPRSKNLTCECKDLIYRMLQPDVSQRLHIDEILSHSWLQPPKPKATSSASFKREGEGKYRAECKLDTKTGLRPDHRPDHKLGAKTQHRLLVVPENENRMEDRLAETSRAKDHHISGAEVGKAST

SEQ ID NO: 6 - protein, 268 aa, Homo sapiens
MEDFLLSNGYQLGKTIGEGTYSKVKEAFSKKHQRKVAIKVIDKMGGPEEFIQRFLPRELQIVRTLDHKNIIQVYEMLESADGKICLVMELAEGGDVFDCVLNGGPLPESRAKALFRQMVEAIRYCHGCGVAHRDLKCENALLQGFNLKLTDFGFAKVLPKSHRELSQTFCGSTAYAAPEVLQGIPHDSKKGDVWSMGVVLYVMLCASLPFDDTDIPKMLWQQQKGVSFPTHLSISADCQDLLKRLLEPDMILRPSIEEVSWHPWLAST

SEQ ID NO: 7 - DNA, 1779 bp, Homo sapiens
atggcgagcgtggtggtgaagacgatctggcagtccaaagagatccatgaggccggggacacccccacgggggtggagagctgctcccagctagtcccagaggctccccggagggtgaccagccgggccaaggggatcccgaagaaaaagaaggccgtgtcgttccacggggtggagccccagatgtcccatcagcccatgcactggtgcctgaacctcaaacggtcctcggcctgcaccaacgtgtcactgctcaacctggccgccatggagcccactgactccacggggacagactccacagtggaagacctcagcggccaactcacactggctgggccccctgcctcccctaccctaccctgggatccggatgacgcagacatcacggaaatcctgagtggggtcaacagtggattggtccgcgccaaagactccatcaccagcttgaaggaaaagaccaaccgggttaaccagcacgtgcagtctctgcagagcgagtgttctgtgctgagcgagaatctggagggaaggcggcaagaggcagaagagttggaggggtactgcattcaactcaaggagaactgctggaaggtgacccggtctgtggaagatgctgaaatcaaaaccaacgtcttgaagcagaattctgccctgctggaggagaagttgcgctacctccagcagcagctgcaggatgagacgccgcgacggcaggaggccgagctgcaggagccggaggagaagcaggagccggaggagaagcaggagccggaggagaagcagaagccggaggctggcctctcctggaacagcctgggccccgccgccacgtcccagggctgccccggcccgccagggagtcccgacaaaccctcgcggccacacggcctggtccccgcaggctggggaatggggcctcgggctggcgagggcccctacgtgagcgagcaggaattgcagaagctgttcaccggcatcgaagagctgaggagagaggtgtcctcactgaccgcccggtggcatcaggaggagggggcggtgcaggaagccctgcggctgctcgggggcctgggcggcagggtcgacggcttcctaggccagtgggagcgggcacagcgcgaacaggcacagacggcgcggggcttgcaggagctgcgaggtcgggcggatgagctgtgcaccatggtggagcggtcagcagtgtctgtggcttcactgaggagcgaactggaggggctgggcccactgaaacccattctggaggagttcgggcggcaatttcagaactctcgaagagggcctgacctctccatgaacctggatcggtcccaccaaggcaactgtgcccgctgtgccagccaggggtcgcagttgtctacggagtccctgcagcagctgctggaccgagcactgacctcactagtggacgaggtgaagcagaggggcctgactcctgcctgtcccagctgtcagaggctacacaagaagattctggagctggagcgccaggccttagccaaacacgtcagggcagaggccctgagctccacccttcggctggcccaagacgaggccctgcgggccaagaacctactgctgacagacaagatgaagccagaggagaagatggccactctggaccatctacacttgaagatgtgctccctccacgatcatctcagcaacctgccacttgaggggtccacgggaacaatggggggaggcagcagtgcaggaacccccccaaaacaggggggctcagcccctgaacaataa

SEQ ID NO: 8 - protein, 592 aa, Homo sapiens
MASVVVKTIWQSKEIHEAGDTPTGVESCSQLVPEAPRRVTSRAKGIPKKKKAVSFHGVEPQMSHQPMHWCLNLKRSSACTNVSLLNLAAMEPTDSTGTDSTVEDLSGQLTLAGPPASPTLPWDPDDADITEILSGVNSGLVRAKDSITSLKEKTNRVNQHVQSLQSECSVLSENLEGRRQEAEELEGYCIQLKENCWKVTRSVEDAEIKTNVLKQNSALLEEKLRYLQQQLQDETPRRQEAELQEPEEKQEPEEKQEPEEKQKPEAGLSWNSLGPAATSQGCPGPPGSPDKPSRPHGLVPAGWGMGPRAGEGPYVSEQELQKLFTGIEELRREVSSLTARWHQEEGAVQEALRLLGGLGGRVDGFLGQWERAQREQAQTARGLQELRGRADELCTMVERSAVSVASLRSELEGLGPLKPILEEFGRQFQNSRRGPDLSMNLDRSHQGNCARCASQGSQLSTESLQQLLDRALTSLVDEVKQRGLTPACPSCQRLHKKILELERQALAKHVRAEALSSTLRLAQDEALRAKNLLLTDKMKPEEKMATLDHLHLKMCSLHDHLSNLPLEGSTGTMGGGSSAGTPPKQGGSAPEQ

SEQ ID NO: 9 - protein, 268 aa, Mus musculus
MEDFLLSNGYQLGKTIGEGTYSKVKEAFSKKHQRKVAIKIIDKMGGPEEFIQRFLPRELQIVRTLDHKNIIQVYEMLESADGKIYLVMELAEGGDVFDCVLNGGPLPESRAKALFRQMVEAIRYCHGCGVAHRDLKCENALLQGFNLKLTDFGFAKVLPKSRRELSQTFCGSTAYAAPEVLQGIPHDSKKGDVWSMGVVLYVMLCASLPFDDTDIPKMLWQQQKGVSFPTHLGISTECQDLLKRLLEPDMILRPSIEEVSWHPWLAST

SEQ ID NO: 10 - DNA, 1020 bp, Mus musculus
gcacgagatgttctagccctggaggcagctgtgaatgaagtccttggggggaaaagaagcaggccgagggcgatggtggagtagagctgcctcgcagaggcagcatgagctgagagggtgacaagaaggaggcgctacacagcatggaggactttctactctccaatgggtatcagctgggcaagaccattggggaagggacctactcaaaagtcaaagaagcattttccaaaaaacatcaacgaaaagtggcaattaaaattatagacaagatgggagggccagaagagtttatccagagattcctgcctcgtgagctccagattgtccgtaccctggaccacaaaaacatcatccaggtgtatgagatgctggagtcagcagatggaaaaatctacctggtgatggagctggctgagggaggggatgtctttgactgtgtgctgaacggagggccacttcccgagagccgggccaaggccctcttccgccagatggttgaggctattcgctattgccatggctgtggcgtggcccaccgggaccttaagtgtgagaacgccttgttgcagggcttcaacctgaagctgaccgactttggctttgccaaggtgctacccaagtcacgcagggagctgagccagaccttctgtggcagcacagcctatgccgcccctgaggtgctacagggcataccccatgatagcaagaaaggtgatgtctggagcatgggtgtggtcctgtatgtaatgctctgtgcaagtctaccttttgacgacacagatatccccaagatgctgtggcagcagcagaagggggtgtccttccccactcatttgggcatctcaaccgaatgccaggacctgctcaagcggctcctggaaccagacatgatactccggccttcaatcgaagaagttagttggcacccatggctagcaagcacttgataaaagcaatggcaagtcctccccaataaagtagggggagaaagcaaactgaaaaaaaaaaaaaaaaaaa

SEQ ID NO: 11 - DNA, 24 bp, Artificial Sequence
  synthetic construct
cgtggatccawaggaccasacrtc

SEQ ID NO: 12 - DNA, 20 bp, Artificial Sequence
  synthetic construct
attcggatccacmgngayyt

SEQ ID NO: 13 - DNA, 24 bp, Artificial Sequence
  synthetic construct
catcacctttcttgctatcatggg

SEQ ID NO: 14 - DNA, 21 bp, Artificial Sequence
  synthetic construct
tgtgagaacgccttgttgcag

SEQ ID NO: 15 - DNA, 23 bp, Artificial Sequence
  synthetic construct
gacatcaccttttttgctatcgt

SEQ ID NO: 16 - DNA, 27 bp, Artificial Sequence
  synthetic construct
ccatcctaatacgactcactatagggc

SEQ ID NO: 17 - DNA, 24 bp, Artificial Sequence
  synthetic construct
gcaggtgagaatgttctaacgctg

SEQ ID NO: 18 - DNA, 24 bp, Artificial Sequence
  synthetic construct
tctccccctactttattggagagc

SEQ ID NO: 19 - DNA, 1467 bp, homo sapiens
acaaagcagggaggctagggtggccccggggcggggtcccggacccccaccctaaggcggagtgacgcccgcagtcacttcacaaggcaaaaattgttacggggcaataaaaggcacaacagcggccaatgtctggcagtgggcacatgggggtgcgggggtgtaggtgccaagcgccatggcttagacccgagattggagtccggccgccccccgacagcagccgcctcctgccccccgtgcgccctaggcgccaccatgtcgggagacaaacttctgagcgaactcggttataagctgggccgcacaattggagagggcagctactccaaggtgaaggtggccacatccaagaagtacaagggtaccgtggccatcaaggtggtggaccggcggcgagcgcccccggacttcgtcaacaagttcctgccgcgagagctgtccatcctgcggggcgtgcgacacccgcacatcgtgcacgtcttcgagttcatcgaggtgtgcaacgggaaactgtacatcgtgatggaagcggccgccaccgacctgctgcaagccgtgcagcgcaacgggcgcatccccggagttcaggcgcgcgacctctttgcgcagatcgccggcgccgtgcgctacctgcacgatcatcacctggtgcaccgcgacctcaagtgcgaaaacgtgctgctgagcccggacgagcgccgcgtcaagctcaccgacttcggcttcggccgccaggcccatggctacccagacctgagcaccacctactgcggctcagccgcctacgcgtcacccgaggtgctcctgggcatcccctacgaccccaagaagtacgatgtgtggagcatgggcgtcgtgctctacgtcatggtcaccgggtgcatgcccttcgacgactcggacatcgccggcctgccccggcgccagaaacgcggcgtgctctatcccgaaggcctcgagctgtccgagcgctgcaaggccctgatcgccgagctgctgcagttcagcccgtccgccaggccctccgcgggccaggtagcgcgcaactgctggctgcgcgccggggactccggctagaagccgggtggttccagccattcctgcagccaagggcactgggccagggcggcgcacgcgcaagaggcgcgcttcgagggaatatgcgaagctgccgcgtgctgctgcacatgcgctttttcccttccgcttccctccctttcttcccacgggggagtccgcagttgcccttgttcggaatccacgttccccgcgatcccgggagctggaggcgcatgcgcatccgcgattccctgcgaccaggccccgagagggcgagaccagaggggacggaagcattgcgcctgcgcggaactctcagcctctgcgcggagggcgtcccttcccaaccagccgtgggtgccaggttcccggttggaacctgcaataaactcgctgttcctcgca

SEQ ID NO: 20 - protein, 273 aa, homo sapiens
MSGDKLLSELGYKLGRTIGEGSYSKVKVATSKKYKGTVAIKVVDRRRAPPDFVNKFLPRELSILRGVRHPHIVHVFEFIEVCNGKLYIVMEAAATDLLQAVQRNGRIPGVQARDLFAQIAGAVRYLHDHHLVHRDLKCENVLLSPDERRVKLTDFGFGRQAHGYPDLSTTYCGSAAYASPEVLLGIPYDPKKYDVWSMGVVLYVMVTGCMPFDDSDIAGLPRRQKRGVLYPEGLELSERCKALIAELLQFSPSARPSAGQVARNCWLRAGDSG

SEQ ID NO: 21 - DNA, 18 bp, Homo sapiens
gcccctaggtggatgagg

SEQ ID NO: 22 - DNA, 19 bp, Homo sapiens
tcacgctctggggggagta

SEQ ID NO: 23 - DNA, 20 bp, Homo sapiens
gggttcctacgcaaaagtca

SEQ ID NO: 24 - DNA, 20 bp, Homo sapiens
gttttcttgcggtcgatgat

SEQ ID NO: 25 - DNA, 20 bp, Homo sapiens
ggggaagggacctactcaaa

SEQ ID NO: 26 - DNA, 20 bp, Homo sapiens
gtccagggtacggacgattt

SEQ ID NO: 27 - DNA, 19 bp, Homo sapiens
tacgcgtcacccgagtgct

SEQ ID NO: 28 - DNA, 18 bp, Homo sapiens
acgcccatgctccacaca

SEQ ID NO: 29 - DNA, 19 bp, Homo sapiens
gctgagcgagaatctggag

SEQ ID NO: 30 - DNA, 20 bp, Homo sapiens
ttcagcatcttccacagacc

SEQ ID NO: 31 - DNA, 20 bp, Homo sapiens
ggattcaaatgaggctccaa

SEQ ID NO: 32 - DNA, 19 bp, Homo sapiens
tggaggtagcgcagcttct

SEQ ID NO: 33 - DNA, 20 bp, Homo sapiens
atcatcagcaatgcctcctg

SEQ ID NO: 34 - DNA, 20 bp, Homo sapiens
atggcatggactgtggtcat

SEQ ID NO: 35 - DNA, 1327 bp, mus musculus
ggtgccaagcaccatgggttaggcccgagattggagtccggccgccccccgacagcagccgcctcctgctccccgcgcgccccgggcgccaccatgtcgggcgacaaactcctgagcgaacttggctataagctgggccgcacgataggtgagggcagctactccaaggtgaaagtggccacctccaagaagtataaagggacggtggccatcaaggtggtggaccgccggcgagcgcctccggacttcgtcaacaagttcctgcctcgagagctgtccatcctgcggggagtacggcacccgcacatcgtacacgttttcgagttcatcgaagtgtgcaatgggaagctgtacatcgtgatggaggcagcagccaccgacctgctgcaggcagtgcagcgcaacgggcgcatcccggggtcgcaggcgcgcgaactcttctcgcagatcgcgggcgccgtgcgctacctgcacgaccaccacctggtgcaccgcgacctcaagtgcgaaaacgtgctgctaagccctgacgagcgccgcgtcaagatcacggatttcggtttcggccgccaggcgcacggctacccagacctgagcaccacctactgcggctcggccgcgtatgcgtcacctgaggtgctcctgggcatcccctacgaccctaagaaatacgacgtgtggagcctgggcgttgtgctctacgtcatggtcaccgggtgcatgcccttcgacgactcggacattgctggcctaccccggcgccagaagcgcggcgtgctctaccctgacggcctcgaactgtcggaacgatgcaaatccttgatcgccgagctgctacagttcagcccgtctgcccgtccctcggcgggccaggtggcgcgcaatggctggctgcgggccggggactccggctaggagccggggttctcgctgttcccgccgcaccaggcacagagccagggcggcgaacgcgcgaagggcgagcttcgggaaacccgcgaagtcgccgcgcgccactgcgcacgcgtcctgtcccttccccttcccccacggcggcggccctctgaggcctgctgttgggttctgattcctcctgcacaaactgattgctggagagcgcatgcgcatcctcgatttctggcgagaaggccccgggaggggcgcaaagagaagagagagaagcattgcgcctgcgcagaatgagctgttgcacggtgggttgccatgctacagagaaaccgtgggccagccgtgcacgctaagttccaggttggaacctgcaataaattcgctcttccttgcaaaaaaaaaaaaaaaaaaaaaaaaa

SEQ ID NO: 36 - DNA, 395 bp, mus musculus
tcgagttcatcgaagtgtgcaatgggaagctgtacatcgtgatggaggcagcagccaccgacctgctgcaggcagtgcagcgcaacgggcgcatcccggggtcgcaggcgcgcgaactcttctcgcagatcgcgggcgccgtgcgctacctgcacgaccaccacctggtgcaccgcgacctcaagtgcgaaaacgtgctgctaagccctgacgagcgccgcgtcaagatcacggatttcggtttcggccgccaggcgcacggctacccagacctgagcaccacctactgcggctcggccgcgtatgcgtcacctgaggtgctcctgggcatcccctacgaccctaagaaatacgacgtgtggagcctgggcgttgtgctctacgtcatggtcacc

SEQ ID NO: 37 - DNA, 20 bp, mus musculus
tcgagttcatcgaagtgtgc

SEQ ID NO: 38 - DNA, 20 bp, mus musculus
ggtgaccatgacgtagagca

(19) United States
(12) Patent Application Publication
(10) Pub. No.: US 2005/0032146 A1
(43) Pub. Date: Feb. 10, 2005

(54) Tssk4: a human testis specific serine/threonine kinase

(76) Inventors: John C. Herr, Charlottesville, VA (US); Pablo E. Visconti, Amherst, MA (US); Andrea Wagenfeld, Berlin (DE); Michael Coppola, Charlottesville, VA (US); Zhonglin Hao, Charlottesville, VA (US); Soumya Vemuganti, Charlottesville, VA (US)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/754,829

(22) Filed: Jan. 8, 2004

Related U.S. Application Data
(60) Provisional application No. 60/493,401, filed on Aug. 7, 2003.

(51) Int. Cl. C12Q 1/48

(52) U.S. Cl. 435/15

(57) ABSTRACT

The present invention relates to a family of testis specific kinases (the tssk family), nucleic acid sequences encoding those kinases and antibodies against the kinases. The invention is further directed to the use of the tssk 4 kinase and other members of the tssk kinase family as targets for isolating specific inhibitors or antagonists of tssk kinase activity. Such inhibitors are anticipated to have use as contraceptive agents.

### RELATED APPLICATIONS

This application claims priority under 35 USC § 19(e) to U.S. Provisional Application Ser. Nos. 60/493,401, filed Aug. 7, 2003, the disclosure of which is incorporated herein by reference.

### US GOVERNMENT RIGHTS

This invention was made with United States Government support under Grant Nos. HD 38082, and U54 29099, awarded by National Institutes of Health. The United States Government has certain rights in the invention.

### BACKGROUND

Spermatogenesis, the process in which functional sperm cells are produced in the testis, involves specific interaction between the developing germ cells and their supporting Sertoli cells as well as hormonal regulation by the androgen-producing Leydig cells. The general organization of spermatogenesis is essentially the same in all mammals and can be divided into three distinct phases: 1) The initial phase is the proliferative or spermatogonial phase during which spermatogonia undergo mitotic division and generate a pool of spermatocytes; 2) the meiotic phase, that yields the haploid spermatids; and 3) spermiogenesis whereby each round spermatid differentiates into a spermatozoon. Although the molecular mechanisms regulating the first two phases have been relatively well characterized, the molecular basis of spermiogenesis is largely unknown.

Mammalian spermiogenesis, the postmeiotic phase of spermatogenesis, is characterized by dramatic morphological changes that occur in the haploid spermatid. Some of these changes include the formation of the acrosome and its contents, the condensation and reorganization of the chromatin, the elongation and species-specific reshaping of the cell, and the assembly of the flagellum. These events result from changes in both gene transcription and protein translation that occurs during this developmental period. Some of the proteins translated in the haploid spermatid will remain in the morphologically mature sperm after it leaves the testis. Taking this into consideration, proteins that are synthesized during spermatogenesis might be necessary for spermatid differentiation and/or for sperm function during fertilization.

One aspect of the present invention relates to signaling events in mammalian sperm that regulate the functions of this highly differentiated cell. More particularly, in one embodiment the invention relates to signal transduction that modulates the acquisition of sperm fertilizing capacity. After ejaculation, sperm are able to move actively but lack fertilizing competence. They acquire the ability to fertilize in the female genital tract in a time-dependent process called capacitation. Capacitation has been demonstrated to be accompanied by phosphorylation of several proteins on both serine/threonine and tyrosine residues, and that protein tyrosine phosphorylation is regulated downstream by a cAMP/PKA pathway that involves the crosstalk between these two signaling pathways. With the exception of PKA, the other kinase(s) involved in the regulation of capacitation are still unknown.

Examples of testis-specific kinases include the recently described mouse genes, tssk 1, 2 and 3 (Bielke et al., 1994, Gene 139, 235-9; Kueng et al, 1997, J Cell Biol 139, 1851-9; Zuercher et al., 2000, Mech Dev 93, 175-7). Using a combination of two yeast hybrid technology and coimmunoprecipitation, Kueng et al. (1997) found that mouse tssk 1 and 2 bind and phosphorylate a protein of 54 Kda. That 54 Kda protein represents the tssk substrate, designated as tsks. The tsks protein is also testis-abundant and its developmental expression suggests that it is postmeiotically expressed in germ cells. The mouse cDNA sequence of the tssk substrate was previously reported (Kueng et al., 1997) and was used to search the EST data base. A human EST homologue AL041339 was found and used to generate sense and antisense primers for obtaining the full length clone by 5′ and 3′ RACE using human testis marathon ready cDNA (Clontech, Inc.). The full length nucleotide sequence of human tsks is provided as SEQ ID NO: 7 and the deduced protein sequence is provided as SEQ ID NO: 8.

The function of the tssk kinase family is unknown. However, since the members of this family are expressed postmeiotically during spermiogenesis, it is hypothesized that they have a role in germ cell differentiation, or later on in sperm function. Therefore, it is anticipated that compounds that interfere with the function of this kinase family could be utilized as contraceptive agents.

Despite the availability of a range of contraceptive methods, over 50% of pregnancies are unintended worldwide and in the United States. Thus, there is a critical need for contraception that better fits the diverse needs of women and men and takes into consideration different ethnic, cultural and religious values. Except for the use of condoms or vasectomy, the availability of contraceptive methods for men is very limited.

In accordance with one embodiment tssk 4 serves as a target for the development of novel drugs, including the identification of novel contraceptive agents. Finally, if the tssk kinases remain in the sperm after spermatogenesis and have a role in sperm physiology, design of specific tssk kinase inhibitors could be used both in male and female in order to prevent fertilization.

### SUMMARY OF VARIOUS EMBODIMENTS OF THE INVENTION

The present invention is directed to a family of sperm specific kinases (tssk) genes, their respective encoded proteins and antibodies against those proteins. More particularly, the present invention is directed to human kinase 4 (tssk4) and the use of that kinase to identify agonists or antagonists of tssk 4 kinase activity. Antagonists of tssk 4 activity are anticipated to have utility as contraceptive agents. The present invention also encompasses antibodies specific for tssk 4 and the use of such antibodies as therapeutic and diagnostic tools.

### BRIEF DESCRIPTION OF THE DRAWINGS

FIG. 1. Comparison between the amino acid sequences of human tssk 1 (SEQ ID NO: 4), human tssk 2 (SEQ ID NO: 5), human tssk 3 (SEQ ID NO: 6) and human tssk 4 (SEQ ID NO: 20). As seen in FIG. 1 the amino acid sequences between the four proteins diverge near the carboxy terminus.

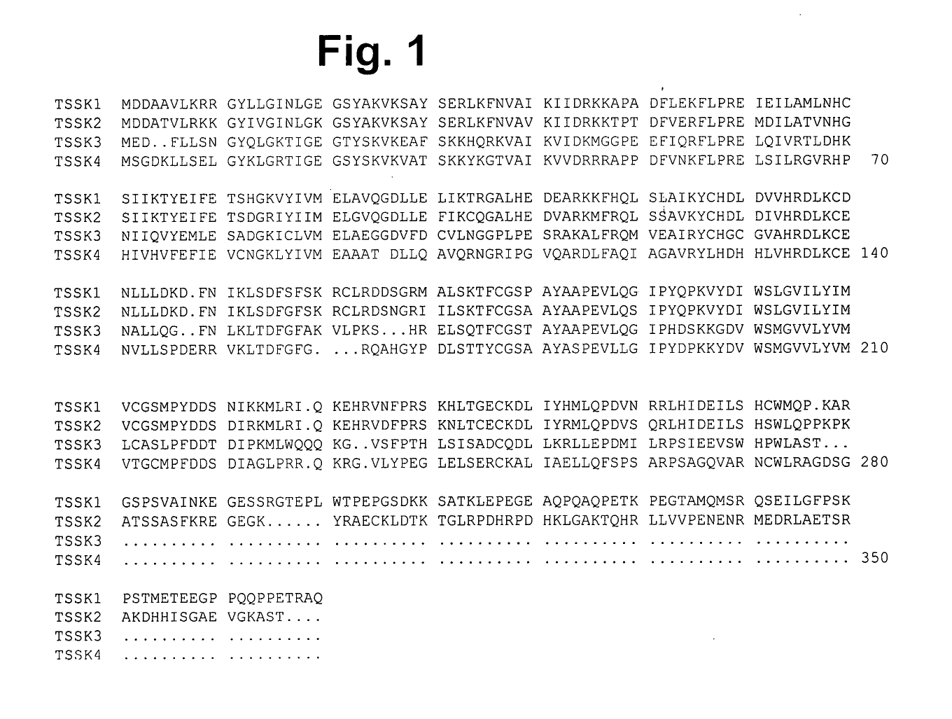

FIG. 2. Graphic representation of data generated from real time PCR of cDNAs prepared from various human tissues and conducted using primers specific for tssk1.

FIG. 3. Graphic representation of data generated from real time PCR of cDNAs prepared from various human tissues and conducted using primers specific for tssk2.

FIG. 4. Graphic representation of data generated from real time PCR of cDNAs prepared from various human tissues and conducted using primers specific for tssk3.

FIG. 5. Graphic representation of data generated from real time PCR of cDNAs prepared from various human tissues and conducted using primers specific for tssk4.

FIG. 6. In situ hybridization of mouse testis tissues using a tssk 4 probe.

FIG. 7. Northern blot of mouse poly-A mRNA isolated from various tissues and probed using a 1.4 kilobase mouse TSSK 4 cDNA probe labeled with 32P-dCTP by random priming.

### DETAILED DESCRIPTION OF EMBODIMENTS

Definitions

In describing and claiming the invention, the following terminology will be used in accordance with the definitions set forth below.

As used herein, the term “purified” and like terms relate to an enrichment of a molecule or compound relative to other components normally associated with the molecule or compound in a native enviromnent. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A polylinker is a nucleic acid sequence that comprises a series of three or more closely spaced restriction endonuclease recognitions sequences.

“Operably linked” refers to a juxtaposition wherein the components are configured so as to perform their usual function. Thus, control sequences or promoters operably linked to a coding sequence are capable of effecting the expression of the coding sequence.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:
1. peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂-S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;
2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;
3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for trytophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

As used herein, the term “antibody” refers to a polyclonal or 30 monoclonal antibody or a binding fragment thereof such as Fab, F(ab′)2 and Fv fragments.

As used herein, the term “biologically active fragments” or “bioactive fragment” of a tssk polypeptide encompasses natural or synthetic portions of the full-length protein that are capable of specific binding to their natural ligand.

The term “non-native promoter” as used herein refers to any promoter that has been operably linked to a coding sequence wherein the coding sequence and the promoter are not naturally associated (i.e. a recombinant promoter/coding sequence construct).

As used herein, a transgenic cell is any cell that comprises a nucleic acid sequence that has been introduced into the cell in a manner that allows expression of a gene encoded by the introduced nucleic acid sequence.

As used herein “an inhibitor of tssk 4 kinase activity” or “tssk 4 inhibitor” is intended to include any compound, composition or environmental factor that decreases overall tssk kinase activity without significantly impacting the activity of non-tssk kinases. Thus inhibitors include factors that decrease the specific activity of the kinase as well as factors that decrease the number of active kinase molecules available for the reaction (i.e. transcriptional and translational inhibitors for in vivo situations). The term “tssk 4 specific inhibitor” refers to a tssk 4 inhibitor that only decreases the activity of tssk 4 kinase and not other tssk family members or other kinases.

### Embodiments

The present invention is directed to a family of kinases (the tssk kinase family) that are testis abundant and expressed predominantly if not exclusively in the male germ cells of humans and mice. More particularly the present invention is directed to tssk 4 and the use of that protein to prepare and isolate compounds that can be used as diagnostic and contraceptive agents.

The developmental expression pattern of the tssk kinases, as well as the general relevance of kinases in physiological processes has led applicants to believe that this family of testis-abundant kinases has a role in spermatogenesis. The finding that the tssk kinase family and one of the putative substrates are expressed at the same time during spermatogenesis is relevant to the potential use of these proteins as contraceptive targets. Accordingly, one aspect of the present invention is directed to the isolation of the human tssk homologs and their use in isolating agents that inhibit tssk kinase activity. Such inhibitors can then be used as contraceptive agents to inhibit fertilization. In accordance with one embodiment, the sperm-specific tssk gene products, including tssk 1, tssk 2, tssk 3 and tssk 4 are used to screen for specific inhibitors of tssk kinase activity and these inhibitors will be used either alone or in conjunction with other contraceptive agents to prevent unintended pregnancies. More particularly, in one embodiment, tssk 4 is used as a target for identifying compounds that specifically inhibit tssk activity and thus serve as contraceptive agents. Advantageously, the unique sequence of the members of the tssk kinase family supports the likelihood of finding specific inhibitors for their activity that do not significantly inhibit the activity of non-tssk kinases. In addition to screening for tssk specific inhibitors that inhibit all tssk kinase activity, the present invention also encompasses screening for inhibitors that inhibit some of the tssk family members (e.g. a tssk 1/tssk 3/tssk 4 specific inhibitor) or that only inhibit the activity of a single tssk family member (e.g. a tssk 4 specific inhibitor).

Since sperm are transcriptionally and translationally inactive, cloning and characterizing candidate sperm protein kinases at the molecular level, requires the use of RNA isolated from male germ cells. RNA transcripts expressed in the male germ cell lineage might ultimately be important in sperm function; however, it is also possible that such transcripts function during testicular spermatogenesis. Using this methodology a unique cDNA was cloned from mouse male germ cells, and the cDNA was determined to encode a putative protein kinase of the ser/thr protein kinase subfamily. That kinase (tssk 3b) is specifically expressed postmeiotically in murine male germ cells. The amino acid sequence and nucleic acid sequence of tssk 3b is provided as SEQ ID NO 9 and SEQ ID NO: 10, respectively.

The human homologue of tssk3b has also been cloned (and designated human tssk 3) and exhibits 98% homology to the putative mouse protein at the protein level. The nucleotide sequence and amino acid sequence of human tssk 3 is provided as SEQ ID NO 3 and SEQ ID NO: 6, respectively. Recently, a similar mouse protein kinase was described and identified as a testis-specific serine kinase 3 (mouse tssk 3) (Zuercher et al., 2000, Mech Dev 93, 175-7), a member of a small family of testis-specific protein kinases (Bielke et al., 1994, Gene 139, 235-9; Kueng et al., 1997, J Cell Biol 139, 1851-9). Despite this homology, both the human tssk 3 and the mouse and the mouse tssk 3b cDNAs cloned and described in the present application demonstrate differences with mouse tssk 3 in several amino acids.

Using the predicted mouse tssk 1 and 2 amino acid sequences (as described by Kueng et al., 1997) for the purposes of searching the gene bank, a genome sequence of both mouse tssk 1 and tssk 2 was obtained. The 3′ end and 5′ end of the coding region of these genomic mouse genes were then used to design sense and antisense primers, respectively, and used to isolate the human homologs using PCR technology (see Example 2). The amplified cDNA fragments were cloned using the TOPO TA cloning kit (Invitrogen) and sequenced. An amplified human tssk 1 gene was approximately 1.3 kb in size and the isolated human tssk 2 gene was approximately 1.2 kb in size. The nucleic acid sequence and amino acid sequence of human tssk 1 is provided as SEQ ID NO: 1 and SEQ ID NO: 4, respectively. The nucleic acid sequence and amino acid sequence of human tssk 2 is provided as SEQ ID NO: 2 and SEQ ID NO: 5, respectively. A fourth member of the human tssk family has now been discovered and is designated tssk 4. The nucleic acid and amino acid sequence of tssk 4 is provided as SEQ ID NO: 19 and SEQ ID NO: 20, respectively.

In accordance with one embodiment of the present invention a purified polypeptide is provided comprising the amino acid sequence of SEQ ID NO: 4, SEQ ID NO: 5, SEQ ID NO: 6 or SEQ ID NO: 20, or an amino acid sequence that differs from SEQ ID NO: 4, SEQ ID NO: 5, SEQ ID NO: 6 or SEQ ID NO: 20 by one or more conservative amino acid substitutions. In another embodiment the purified polypeptide comprises an amino acid sequence that differs from SEQ ID NO: 4, SEQ ID NO: 5, SEQ ID NO: 6 or SEQ ID NO: 20 by less than 5 conservative amino acid substitutions, and in a further embodiment, by 2 or less conservative amino acid substitutions. In one embodiment the purified polypeptide comprises the amino acid sequence of SEQ ID NO: 4, SEQ ID NO: 5, SEQ ID NO: 6 or SEQ ID NO: 20.

The polypeptides of the present invention may include additional amino acid sequences to assist in the stabilization and/or purification of recombinantly produced polypeptides. These additional sequences may include intra- or inter-cellular targeting peptides or various peptide tags known to those skilled in the art. In one embodiment, the purified polypeptide comprises an amino acid sequence selected from the group consisting of SEQ ID NO: 4, SEQ ID NO: 5, SEQ ID NO: 6 and SEQ ID NO: 20 and a peptide tag, wherein the peptide tag is linked to the tssk peptide sequence. Suitable expression vectors for expressing such fusion proteins and suitable peptide tags are known to those skilled in the art and commercially available. In one embodiment the tag comprises a His tag (see Example 4). In another embodiment the purified polypeptide comprises the amino acid sequence of SEQ ID NO: 20 linked to a peptide tag.

In another embodiment, the present invention is directed to a purified polypeptide that comprises an amino acid fragment of a tssk polypeptide. More particularly the tssk polypeptide fragment consists of natural or synthetic portions of a full-length polypeptide selected from the group consisting of SEQ ID NO: 4, SEQ ID NO: 5, SEQ ID NO: 6, SEQ ID NO: 9 and SEQ ID NO: 20 that are capable of specific binding to their natural ligand. In one embodiment the human tssk fragment retains its ability to bind to tsks.

The present invention also encompasses nucleic acid sequences that encode human tssk. In one embodiment a nucleic acid sequence is provided comprising the sequence of SEQ ID NO: 1, SEQ ID NO: 2, SEQ ID NO: 3, SEQ ID NO: 10, SEQ ID NO: 19 or fragments thereof. In another embodiment a purified nucleic acid sequence is provided, selected from the group consisting of SEQ ID NO: 1, SEQ ID NO: 2, SEQ ID NO: 3 and SEQ ID NO: 19.

The present invention is also directed to recombinant human tssk gene constructs. In one embodiment, the recombinant gene construct comprises a non-native promoter operably linked to a nucleic acid sequence selected from the group consisting of SEQ ID NO: 1, SEQ ID NO: 2, SEQ ID NO: 3, SEQ ID NO: 7, SEQ ID NO: 10 and SEQ ID NO: 19. In one embodiment the recombinant gene construct comprises a non-native promoter operably linked to the nucleic acid sequence of SEQ ID NO: 19. The non-native promoter is preferably a strong constitutive promoter that allows for expression in a predetermined host cell. These recombinant gene constructs can be introduced into host cells to produce transgenic cell lines that synthesize the tssk gene products. Host cells can be selected from a wide variety of eukaryotic and prokaryotic organisms, and two preferred host cells are E. coli and yeast cells.

In accordance with one embodiment, a nucleic acid sequence selected from the group consisting of SEQ ID NO: 1, SEQ ID NO: 2, SEQ ID NO: 3 and SEQ ID NO: 19 are inserted into a eukaryotic or prokaryotic expression vector in a manner that operably links the gene sequences to the appropriate regulatory sequences, and human tssk is expressed in the appropriate eukaryotic or prokaryotic cells host cell. In one embodiment the gene construct comprises the nucleic acid sequence of SEQ ID NO: 19 operably linked to a eukaryotic promoter. Suitable eukaryotic host cells and vectors are known to those skilled in the art. The baculovirus system is also suitable for producing transgenic cells and synthesizing the tssk genes of the present invention. One aspect of the present invention is directed to transgenic cell lines that contain recombinant genes that express human tssk 4 and fragments of the human tssk 4 coding sequence. As used herein a transgenic cell is any cell that comprises an exogenously introduced nucleic acid sequence.

In one embodiment the introduced nucleic acid is sufficiently stable in the transgenic cell (i.e. incorporated into the cell's genome, or present in a high copy plasmid) to be passed on to progeny cells. The cells can be propagated in vitro using standard cell culture procedure, or in an alternative embodiment, the host cells are eukaryotic cells and are propagated as part of an animal, including for example, a transgenic animal. In one embodiment the transgenic cell is a human cell and comprises a nucleic acid sequence selected from the group consisting of SEQ ID NO: 1, SEQ ID NO: 2, SEQ ID NO: 3, SEQ ID NO: 7 and SEQ ID NO: 19. In one embodiment the transgenic cell comprises a recombinant nucleic acid sequence, wherein the recombinant nucleic acid sequence comprises SEQ ID NO: 19 or a fragment thereof operably linked to a non-native promoter. The present invention also includes non-human transgenic organisms wherein one or more of the cells of the transgenic organism comprise a recombinant gene that expresses a human tssk product, and more particularly in one embodiment expresses the human tssk 4 sequence.

The present invention also encompasses a method for producing human tssks, including tssk 4. The method comprises the steps of introducing a nucleic acid sequence, comprising a promoter operably linked to a sequence that encodes a human tssk, into a host cell, and culturing the host cell under conditions that allow for expression of the introduced human tssk gene. In one embodiment the promoter is a conditional or inducible promoter, alternatively the promoter may be a tissue specific or temporal restricted promoter (i.e. operably linked genes are only expressed in a specific tissue or at a specific time). The synthesized tssks (e.g. tssk4) can be purified using standard techniques and used in high throughput screens to identify inhibitors of tssk activity, and more specifically inhibitors of tssk 4. Alternatively, in one embodiment the recombinantly produced tssk 4 polypeptides, or fragments thereof are used to generate antibodies against the tssk 4 polypeptides. The recombinantly produced tssk 4 proteins can also be used to obtain crystal structures. Such structures would allow for crystallography analysis that would lead to the design of specific drugs to inhibit tssk 4 function.

Preferably, the nucleic acid sequences encoding the sperm-specific kinase are inserted into a suitable expression vector in a manner that operably links the gene sequences to the appropriate regulatory sequences for expression in the preselected host cell. Suitable host cells, vectors and methods of introducing the DNA constructs into cells are known to those skilled in the art. In particular, nucleic acid sequences encoding the sperm-specific kinase may be added to a cell or cells in vitro or in vivo using delivery mechanisms such as liposomes, viral based vectors, or microinjection.

In accordance with one embodiment a composition is provided comprising a purified peptide having the sequence selected from the group consisting of SEQ ID NO: 4, SEQ ID NO: 5, SEQ ID NO: 6 and SEQ ID NO: 20, or an antigenic fragment thereof. In one embodiment the peptide consists of the sequence of SEQ ID NO: 20. The compositions can be combined with a pharmaceutically acceptable carrier or adjuvants and administered to a mammalian species to induce an immune response.

Another embodiment of the present invention is directed to antibodies specific for the individual tssk isotypes, including tssk 4. In one embodiment the antibody is a monoclonal antibody. The antibodies or antibody fragments of the present invention can be combined with a carrier or diluent to form a composition. In one embodiment, the carrier is a pharmaceutically acceptable carrier. Such carriers and diluents include sterile liquids such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions.

Antibodies to human tssks may be generated using methods that are well known in the art. In accordance with one embodiment an antibody is provided that specifically binds to a polypeptide selected from the group consisting of SEQ ID NO: 4, SEQ ID NO: 5, SEQ ID NO: 6, SEQ ID NO: 8, SEQ ID NO: 9 and SEQ ID NO: 20. In one embodiment antibodies are provided that bind to a polypeptide comprising SEQ ID NO: 20 or an antigenic fragment thereof. The antibodies may be used with or without modification, and may be labeled by joining them, either covalently or non-covalently, with a reporter molecule. In addition, the antibodies can be formulated with standard carriers and optionally labeled to prepare therapeutic or diagnostic compositions.

For the production of antibodies, various host animals, including but not limited to rabbits, mice, rats, etc can be immunized by injection with a tssk4 polypeptide or peptide fragment thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of SLLP 1, SLLP2 or SLLP3 together with genes from a human antibody molecule of appropriate biological activity can be used; such antibodies are within the scope of this invention.

According to the invention, techniques described for the production of single chain antibodies (U.S. Pat. No. 4,946,778) can be adapted to produce egg surface protein-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for tssk epitopes.

Antibody fragments which contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). The foregoing antibodies can be used in methods known in the art relating to the localization and activity of the tssk4 proteins of the invention, e.g., for imaging these proteins, measuring levels thereof in appropriate physiological samples, in diagnostic methods, etc. Antibodies generated in accordance with the present invention may include, but are not limited to, polyclonal, monoclonal, chimeric (i.e “humanized” antibodies), single chain (recombinant), Fab fragments, and fragments produced by a Fab expression library.

The present invention also provides a method for detecting the presence of human tssk. The method comprises the steps of contacting a sample with a labeled antibody that specifically binds to human tssk, removing unbound and non-specific bond material and detecting the presence of the labeled antibody. In one embodiment the labeled compound comprises an antibody that is labeled directly or indirectly (i.e. via a labeled secondary antibody). In particular, the tssk antibodies of the present invention can be used to confirm the expression of tssk as well as its cellular location, or in assays to monitor individuals receiving a tssk inhibitory composition as a means of contraception.

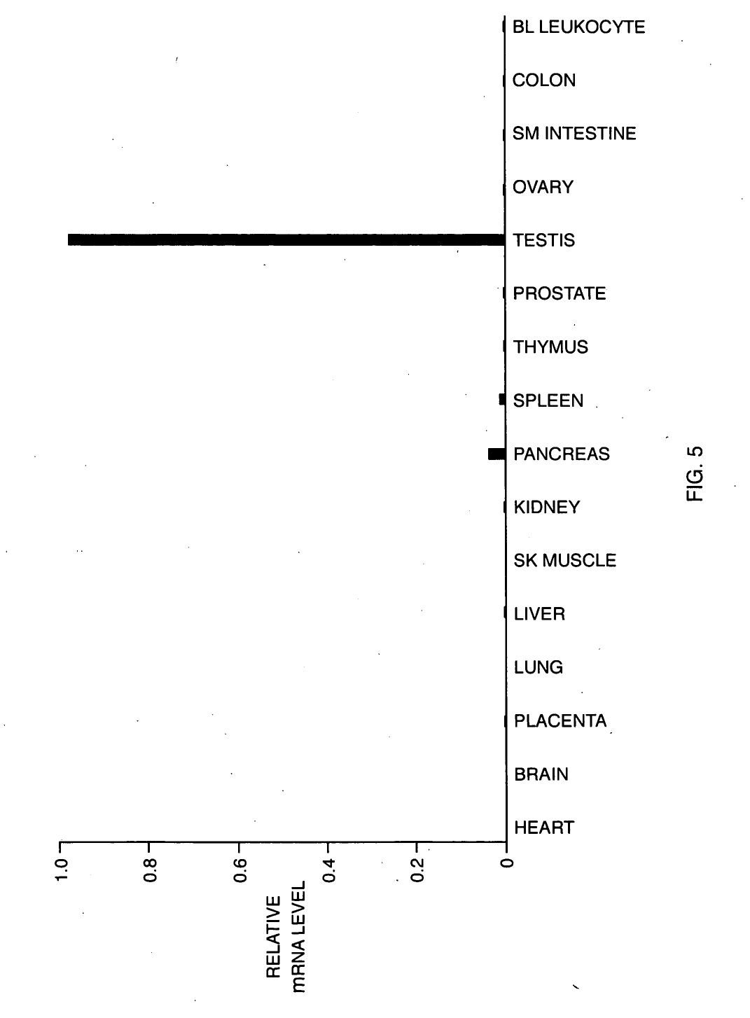

Tssk 4 is demonstrated herein to be highly testis abundant (See FIGS. 5-7), if not exclusively produced in the testis. This makes the tssk 4 kinase an optimal target for the development of drugs that modulate its activity to study tssk's role in spermiogenesis. Furthermore, inhibitors of tssk 4 activity are anticipated to have utility as contraceptive agents. In accordance with one aspect of the present invention, the tssk kinase family, and tssk 4 in particular, is used as a target for the development of novel drugs. Progress in the field of small molecule library generation, using combinatorial chemistry methods coupled to high-throughput screening, has accelerated the search for ideal cell-permeable inhibitors. In addition, structural-based design using crystallographic methods has improved the ability to characterize in detail ligand-protein interaction sites that can be exploited for ligand design.

In one embodiment, the present invention provides methods of screening for agents, small molecules, or proteins that interact with polypeptides comprising the sequence of tssk 1, tssk 2, tssk 3, tssk 4 or bioactive fragments thereof. As used herein, the term “biologically active fragments” or “bioactive fragment” of tssk 1, tssk 2, tssk 3 and tssk 4 encompasses natural or synthetic portions of the native peptides that are capable of specific binding to at least one of the natural ligands of the respective native tssk 1, tssk 2, tssk 3 and tssk 4 polypeptide, including tsks. The invention encompasses both in vivo and in vitro assays to screen small molecules, compounds, recombinant proteins, peptides, nucleic acids, antibodies etc. which bind to or modulate the activity of tssk 1, tssk 2, tssk 3 and tssk 4 and are thus useful as therapeutic or diagnostic markers for fertility.

In one embodiment of the present invention tssk polypeptides, selected from the group consisting of SEQ ID NO: 4, SEQ ID NO: 5, SEQ ID NO: 6 and SEQ ID NO: 20, are used to isolate ligands that bind to tssk under physiological conditions. In one embodiment the sequence of SEQ ID NO: 20, or a bioactive fragment thereof, is used to isolate ligands that bind to tssk 4 under physiological conditions. The screening method comprises the steps of contacting a tssk polypeptide with a mixture of compounds under physiological conditions, removing unbound and non-specifically bound material, and isolating the compounds that remain bound to the tssk polypeptide. Typically, the tssk polypeptide will be bound to a solid support, using standard techniques, to allow for rapid screening of compounds. The solid support can be selected from any surface that has been used to immobilize biological compounds and includes but is not limited to polystyrene, agarose, silica or nitrocellulose. In one embodiment the solid surface comprises functionalized silica or agarose beads. Screening for such compounds can be accomplished using libraries of pharmaceutical agents and standard techniques known to the skilled practitioner.

Ligands that bind to the tssk polypeptides can then be further analyzed for agonists and antagonists activity through the use of an in vitro kinase assay such as that described in Example 7. Inhibitors of tssk kinase activity have potential use as agents that prevent maturation/capacitation of sperm. In accordance with one embodiment, inhibitors of tssk 4 are isolated as potential contraceptive agents. Such inhibitors can be formulated as pharmaceutical compositions and administered to a subject to block spermatogenesis and provide a means for contraception.

In accordance with one embodiment, specific inhibitors of human tssk kinase activity are identified through the use of an in vitro kinase assay that is capable to detecting phosphorylation events. In one embodiment the method of identifying inhibitors of tssk kinase activity comprises combining a labeled source of phosphate with one or more of the human tssk polypeptides in the presence of one or more potential inhibitory compounds. In one embodiment inhibitors of tssk 4 kinase activity are identified by combining a labeled source of phosphate with the human tssk 4 polypeptide (or bioactive fragment thereof) in the presence of one or more potential inhibitory compounds. The reactions are conducted under standard conditions that in the absence of the potential inhibitory compound are suitable for initiating the phosphorylation of the tssk substrate or the tssk enzyme itself.

Decreases in tssk activity can be detected by comparing the amount of phosphorylated tssk substrate in the assay relative to a standard curve plotting kinase activity vs. time. Alternatively an inhibitory decrease in kinase activity can also be detected by conducting a second kinase reaction wherein an in vitro kinase assay composition, comprising a labeled source of phosphate, a tssk substrate and a tssk 4 kinase, is incubated under conditions permissive for kinase activity using identical conditions as that used for the test assay (the assay conducted in the presence to potential inhibitory compounds). The amount of phosphorylated tssk substrate produced by the test assay is then compared to the amount of phosphorylated tssk substrate produced by the second kinase assay (conducted in the absence of the candidate inhibitor) to detect a tssk inhibitory effect of the candidate compound.

Once compound have been identified that inhibit tssk activity, further testing will need to be conducted to isolate those compounds that specifically inhibit tssk activity. In accordance with one embodiment the method of identifying tssk inhibitors further comprises the step of conducting a second reaction wherein the candidate compound is contacted with a control composition wherein the control composition comprises a the candidate compound, a labeled source of phosphate, a kinase substrate and a non-tssk kinase, and determining if the candidate- compound decreases the activity of the non-tssk kinase.

As described in Example 4 the tssk proteins have the property to become autophosphorylated. Therefore by comparing the rate of autophosphorylation that occurs in the presence and absence of the candidate inhibitory compound, specific inhibitory compounds can be identified. In one embodiment a tssk substrate is provided and the assay is based on measuring the rate of phosphorylation of the substrate in the presence and absence of the candidate inhibitory compounds. Preferably large numbers of compounds will screened using high through put techniques to identify tssk specific inhibitory compounds.

In accordance with one embodiment specific inhibitors of tssk kinase activity are identified by providing an in vitro kinase assay composition, wherein the composition comprises a labeled source of phosphate, a tssk substrate and a tssk kinase selected from the group consisting of SEQ ID NO: 4, SEQ ID NO: 5, SEQ ID NO: 6, SEQ ID NO: 9 and SEQ ID NO: 20. The rate of tssk substrate phosphorylation will be determined under controlled conditions in the absence of any inhibitory compounds, and then the identical conditions will be used to measure the rate of phosphorylation of the tssk substrate when the assay is run in the presence of one or more potential inhibitory compounds. Those compounds that decrease the activity of the tssk kinase will be identified and tested to determine if the inhibitory effect is specific to the tssk kinase family.

In one embodiment the method for identifying human tssk inhibitors comprises the steps of providing a labeled source of phosphate, a tssk substrate and a tssk kinase selected from the group consisting of SEQ ID NO: 4, SEQ ID NO: 5, SEQ ID NO: 6 and SEQ ID NO: 20, contacting that composition with one or more potential inhibitory compounds and measuring the rate of phosphorylation. In one embodiment modulators of tssk 4 activity are identified by conducting an in vitro kinase assay, wherein the assay comprises the steps of combining tssk 4 with a labeled source of phosphate (such as γ32P] ATP) and a tssk substrate. In one embodiment the tssk substrate comprises the amino acid sequence of SEQ ID NO: 8. The kinase assay can also be conducted with two or more of human tssk kinases selected from the group consisting of tssk 1, tssk 2, tssk 3, and tssk 4, to identify inhibitors that suppress several of the tssk kinases. It may also be advantageous to identify inhibitors that only suppress certain members of the tssk family, such as inhibitors that decrease tssk 1, tssk 2, and tssk 4 activity, but not tssk 3 or other non-tssk kinases.

One embodiment of the present invention is directed to decreasing the fertility of a male mammal, said method comprising the steps of inhibiting the activity of the tssk kinase family (including tssk 1, tssk 2, tssk 3 and tssk 4). In one embodiment the fertility of a male mammal is decreased by the administration of a pharmaceutical composition that comprises an agent that specifically interferes with tssk activity. In one embodiment the male mammal is a human and the pharmaceutical composition comprises an inhibitor of tssk 4 activity. In one embodiment the composition comprises a tssk 4 specific inhibitor.

In one embodiment the fertility inhibiting composition comprises a chemical entity that specifically inhibits the enzymatic activity of one or more of the tssk kinases as determined by the kinase assay described in Example 7. In another embodiment the inhibitory composition may comprise an antibody against one or more of the tssk kinases or the composition may comprise an antisense or interference RNA that prevents or disrupts the expression of the tssk kinases in an animal. Interference RNA in mammalian systems requires the presence of short interfering RNA (siRNA), which consists of 19-22nt double-stranded RNA molecules, or shRNA, which consists of 19-29nt palindromic sequences connected by loop sequences. Down regulation of gene expression is achieved in a sequence-specific manner by pairing between homologous siRNA and target RNA. A system for the stable expression of siRNA or shRNA was utilized to generate transgenic animals (Hasuwa et al. FEBS Lett 532, 227-30 (2002), Rubinson et al. Nat Genet 33, 401-6 (2003) and Carmell et al. Nat Struct Biol 10, 91-2 (2003)) and can be used in accordance with the present invention to produce animals whose fertility can be regulated. A conditional RNAi-based transgenic system would provide the additional benefit of being able to control the level of gene expression at any given stage during the life of the animal.

### EXAMPLE 1

Isolation of a Novel Mouse Testis Specific Serine/Threonine Kinase

Reverse transcription-polymerase chain reaction (RT-PCR) using degenerate oligonucleotides corresponding to conserved regions present in protein kinases resulted in the isolation of a novel member of the testis-specific serine/threonine kinase. This PCR fragment recognized a 1020 bp transcript in male germ cells by northern blot analysis. Using this fragment as a probe, a full length cDNA was cloned from a mouse mixed germ cell cDNA library. This cDNA has an open reading frame of 804 bases encoding a protein of 268 amino acids. Tissue expression analysis revealed that this protein kinase is developmentally expressed in mouse testicular germ cells and is not present in brain, ovary, kidney, liver or early embryonic cells.

This novel putative serine/threonine protein kinase (SEQ ID NO: 9) is almost identical to tssk 3 (Zuercher et al., 2000, Mech Dev 93, 175-7), a recently described mouse testis-specific protein kinase, with the exception of several base pair deletions that result in a shift in the coding region and an alteration of 22 amino acids (residues 109 to 131). The human homologue of this novel protein kinase (SEQ ID NO: 6) was subsequently cloned and displayed expression exclusively in the testis. Fluorescence in situ hybridization (FISH) using both the human and mouse cDNA clones revealed syntenic localization on chromosomes 1p34-35 and 4E, respectively. Due to the homology with tssk 3 this novel protein kinase is named tssk 3b.

Materials and Methods

Isolation of Spermatogenic Cell Populations

Purified populations of pachytene spermatocytes, round spermatids, and condensing spermatids were prepared from decapsulated testes of adult mice (CD-1; Charles Rivers) by sequential dissociation with collagenase and trypsin-DNase 1 (Bellve et al., 1977; Romrell et al., 1976). The cells were separated into discrete populations by sedimentation velocity at unit gravity in 2-4% BSA gradients in enriched Krebs Ringer Bicarbonate Medium (EKRB) (Bellve et al., 1977; Romrell et al., 1976). The pachytene spermatocyte and round spermatid populations were each at least 85% pure, while the condensing spermatid population was ˜40-50% pure (contaminated primarily with anucleated residual bodies and some round spermatids).

RNA Isolation and Northern Blot Analysis

Total RNA from somatic tissues, testis of mice of defined ages, and isolated spermatogenic cells of adult mice was isolated by homogenizing the cells in 5 M guanidium isothiocyanate, 25 mM sodium citrate, pH 7.2, 0.5% Sarkosyl, and 0.1 M 2-mercaptoethanol (Chirgwin et al., 1979). Lysates were centrifuged over cushions of 5.7 M CsCl, 0.1 M EDTA at 114,000×g at 20° C. overnight. Pellets were resuspended and extracted with phenol:chloroform, and the RNA was precipitated with ethanol. The integrity of the RNA was verified by ethidium bromide staining of the ribosomal RNA on 1% agarose gels. Equivalent amounts of RNA were subjected to electrophoresis in 1.2% agarose gels containing formaldehyde (Sambrook et al., 1989). RNA was transferred to nitrocellulose paper, baked at 80° C. for 2 hr, and prehybridized in 50% formamide, 5× Denhartdt's, 0.1% SDS, 100 μg/ml Torula RNA, 5×SSPE for a minimum of 1 hr at 42° C. The appropriate DNA probe was generated by PCR, radiolabeled with 32p-dCTP by the random-primed method and incubated with the blots (1×10⁶ cpm/ml) in 50% formamide, 5× Denhartdt's, 0.1% SDS, 100 μg/ml Torula RNA, 5×SSPE with 10% dextran sulfate and hybridized at 42° C. overnight. Blots were washed in 2×SSPE, 0.1% SDS and then in 0.1×SSPE, 0.1% SDS, both for 2×10 min at room temperature. After washing, the filters were air-dried and exposed to film at −70° C. with intensifying screens. Northern blots of human tissues were acquired from Clontech and hybridization was performed as described above.

Reverse Transcription-Polymerase Chain Reaction

Reverse transcription-polymerase chain reaction (RT-PCR) was performed using Superscript II from Gibco-BRL according to the manufacturer's instructions, followed by the use of Taq Polymerase from Promega. The PCR conditions were as follows: 1 cycle of 2 min. at 95° C.; 35 cycles of 1 min. at 95° C., 1 min at 55° C. and 1 min at 72° C.; the cycles were finished with 1 cycle of 7 min at 72° C. and then chilled to 4° C. The DNA products were then analyzed on 2% agarose gels. The following degenerate primers were used to target conserved regions of tyrosine kinases: for the reverse transcription step, a degenerate antisense 20-mer corresponding to the coding subregion IX common to the subfamily of tyrosine kinases with the addition of a 5′ Eco RI consensus site for subcloning after three random nucleotides (CGTGGATCCA(A/T)AGGACCA(C/G)AC(A/G)TC; SEQ ID NO: 11); (2) For the PCR step the same primer was used together with a degenerate sense 20-mer corresponding to the subregion VIb common to the subfamily of tyrosine kinases, also a consensus site for Eco RI was introduced after four random nucleotides ATTCGGATCCAC(A/C)G(A/C/T/G)GA(C/T)(C/T)T; SEQ ID NO: 12. After the PCR reaction was completed and the products analyzed, the cDNA was subcloned into a TOPO TA cloning vector according to the manufacturers instructions (In Vitrogen), the Eco RI subcloning consensus sites were not used. Minipreps from 35 positive colonies were prepared using a Qiagen kit and then sequenced using a T7 primer.

Cloning of Mouse Tssk 3b

To clone tssk 3b, the unique sequence obtained by RT-PCR was used to design specific primers against this novel kinase. Primers A2 (antisense: CATCACCTTTCTTGCTATCATGGG; SEQ ID NO: 13) and S2 (sense:(TGTGAGAACGCCTTGTTGCAG; SEQ ID NO: 14) were used to obtained a PCR fragment of 169 bases. This PCR fragment was subsequently radiolabeled with 32p-dCTP by the random-primed method and used as a probe to screen a oligo-dT primed mixed germ cell cDNA library as previously described.

Cloning of the Human Tssk 3b Homologue

Using the predicted mouse tssk 3b amino acid sequence for the purposes of searching the human EST database, entry AI 553938 was pulled out in a BLAST search. This ESTsequence was used to design an antisense primer A3 (GACATCACCTTTTTTGCTATCGT; SEQ ID NO: 15). This primer and an adaptor primer (CCATCCTAATACGACTCACTATAGGGC; SEQ ID NO: 16) were used in 5′ RACE using human testis marathon ready cDNA as the template (Clontech, Inc ). The PCR reaction mix was first heated for 10 min at 94° C. and the mix then subjected to 40 cycles of PCR under the following condition: 94° C. 30 sec, 55° C. 30 sec, 72° C. 2 min. Following these cycles, the mix was incubated at 72° C. for 10 min. AmpTaq Gold (Perkin Elmer) was used in place of conventional Taq polymerase for the PCR. The amplified cDNA fragment was cloned and sequenced using the TOPO TA cloning kit (Invitrogen). The 5′ end of the cDNA sequence obtained following 5′ RACE was used to design a sense primer S3 (GCAGGTGAGAATGTTCTAACGCTG; SEQ ID NO: 17); an antisense primer A4 (TCTCCCCCTACTTTATTGGAGAGC; SEQ ID NO: 18) was based on the 3′ end of AI 553938. These two primers were then used to amplify the full length human tssk 3b homologue from the same library using the conditions described above. The amplified 1.058 kb fragment was excised, cloned and sequenced using the University of Virginia sequencing facility. A C-terminal400 bp fragment of the cDNA library amplified tssk 3b human homologue was used for northern blots of human tissues.

DNA Sequencing and Computer Analysis

All sequencing was performed with the AmpliTaq, FS dye terminator cycle sequencing kit chemistry and the appropriate primers using a 373A DNA sequencer (PE Applied Biosystems, Foster City, Calif.). Ambiguities were resolved by sequencing the opposite strand. DNA and protein sequence analyses were performed using the MacVector ™ (Kodak Scientific Imaging Systems, New Haven, Conn.) and Sequencher™ (Gene Codes Corp., Ann Arbor, Mich.) software programs.

Fluorescence In Situ Hybridization and Chromosomal Mapping

Fluorescence in situ hybridization (FISH) and detection of immunofluorescence were carried out as previously described (Bell et al., 1995, Cytogenet. Cell Genet, 70: 263-267). The 1 kb, mouse cDNA clone, as well as the corresponding 1 kb human homologue, were biotinylated by nick translation in a reaction containing 1 μg DNA, 20 μM each of dA TP, dCTP and dGTP (Perkin Elmer), 1 μM dTTP (Perkin Elmer), 25 mM Tris-HCl, pH 7.5,5 mM MgC12 (Sigma), 10 mM B-mercaptoethanol (Sigma), 10 μM biotin-16-dUTP (Boehringher Mannheim), 2 units DNA polymerase I/DNase I (GIBCO, BRL), and H₂O to a total volume of 50 μl. Probes were denatured and hybridized to metaphase spreads from human peripheral lymphocytes and mouse embryonic fibroblasts, respectively. The hybridized probe was detected with fluorescein-labeled avidin and the signal amplified by the addition of anti-avidin antibody (Oncor) and a second layer of fluorescein-labeled avidin. The chromosome preparations were counterstained with DAPI and observed with a Zeiss Axiophot epiflourescence microscope equipped with a cooled CCD camera (Photometrics, Tucson, Ariz.) operated by a Macintosh computer workstation. Digitized images of DAPI staining and FITC signals were captured, pseudocolored, and merged using Oncor version 1.6 software.

Results

Cloning of Protein Kinases from Murine Male Germ Cells

Mammalian sperm capacitation is accompanied by an increase in protein tyrosine phosphorylation of several proteins. Since mature sperm are not able to synthesize proteins and the presence of RNA in these cells is controversial, in order to identify protein kinases by RT-PCR for the purposes of ultimately deducing their function, RNA was first isolated from a mouse mixed germ cell population. Degenerate primers targeting conserved regions in the subfamily of tyrosine kinases were employed and, following TA cloning, 27 sequences obtained as described in Methods were analyzed and compared with sequences in Gene Bank. Although most of the sequences match known members of the tyrosine kinase subfamily, some of the sequences obtained matched members of the subfamily of ser/thr protein kinases.

Between known members of this subfamily such as B-Rafkinase, a sequence with homology to a novel family of ser/thr protein kinases participating in spermiogenesis was detected (Kueng et al., 1997, J Cell Biol 139, 1851-9). To clone this novel ser/thr kinase, a 169 bases specific PCR fragment corresponding to this sequence was radiolabeled and a mouse mixed germ cell cDNA library was screened as described in Methods. A clone containing a 1.02 kb cDNA insert was obtained (SEQ ID NO: 10) and designated tssk 3b following the nomenclature of Kueng et al. Sequence analysis revealed a single open reading frame of 804 nucleotides encoding a 266 amino acids putative ser/thr protein kinase. The nucleotides flanking the start methionine conform well to Kozak consensus sequence. The 3′-untranslated region displays a polyadenylation signal 21 nucleotides upstream of the poly(A) tail. The sequence contains all the expected conserved domains corresponding to a ser/thr kinase.

Expression Pattern of Murine Tssk 3

The expression pattern of murine tssk 3b was investigated by northern blot analysis of total RNA from different mouse tissues using a tssk 3b specific probe corresponding to the full length transcript of tssk 3b. This probe recognized a single transcript of approximately 1 kb exclusively in the mixed mouse germ cell population prepared as described in Methods. At high exposures it is also possible to distinguish a 1.35 kb transcript also exclusively in germ cells, this transcript was not observed when the mouse germ cell library was screened and could represent a splicing alternative of tssk 3b. To further analyze the expression pattern of tssk 3b in the testis, a northern blot with RNA obtained from purified germ cells of the adult testis was probed. tssk 3b mRNA was found to be expressed postmeiotically in round and condensing spermatids but not in the meiotic pachytene spermatocytes. Mouse testes differentiate at d 11-12 of embryonic development and are populated by primordial germ cells. The first spermatogenic wave is initiated a few days after birth and spermatogonia differentiate to early spermatids before puberty. The differentiation to mature sperm is testosterone dependent and occurs after puberty.

To further investigate whether tssk 3b is expressed postmeiotically, total testis RNA was prepared from mice of different postnatal ages (1,3, 7, 10, 15, 20, 24, 30 and adult) and analyzed by northern blot analysis. Transcription of tssk 3b began between 20 and 24 days after birth confirming a postmeiotic expression of this mRNA.

These results demonstrate that the expression pattern of tssk 3b is similar to that of mouse tssk 1 and tssk 2 (Kueng et al., 1997, J Cell Biol 139, 1851 -9), suggesting that tssk 3 mRNA expression is developmentally regulated and that its expression is stimulated postmeiotically around the onset of spermiogenesis.

To further analyze the expression pattern of tssk 3b, we have performed RT-PCR using specific primers in oocytes, metaphase II-arrested eggs and different stages of preimplantation embryo development. No PCR product was observed at any of these stages under conditions in which the correct sized tssk 3b PCR product could be amplified from mixed germ cell total RNA.

Cloning and expression of the human homologue of tssk 3b

Using the mouse tssk 3 sequence, a human EST from germ cell tumor was identified by a BLAST search of the databases, and used in conjunction with 3′ and 5′ RACE to clone the full length human homologue (SEQ ID NO: 3) from an adapted ligated human testis cDNA library (Clontech) as described in Methods.

To investigate the expression pattern of tssk 3 in human tissues, a tissue northern blot (Clontech) was probed with a C terminal400 bp fragment of the human tssk 3 cDNA. 1 kb and 1.35 kb RNA transcripts were expressed exclusively in the testis. Similar to the mouse case, the 1.35 kb fragment could represent an alternative spliced transcript. Both the mouse tssk 3b and the human homologue tssk 3 of these novel ser/thr kinases have the highest homology (98%) between each other, followed by mouse tssk 3 (92%) mouse tssk 1 and mouse tssk 2 (56%) suggesting that this kinase belongs to the same subfamily of novel ser/thr kinases. As mentioned, tssk 3 and tssk 3b have a very high homology (92%), the difference between these two sequences is restricted to a stretch of 22 aminoacids (residues 109 to 131 ). When this stretch is analyzed at the nucleotide level, three base pair deletions were observed in the mouse tssk 3 sequence that resulted in a shift in the coding region and an alteration of the aforementioned 22 amino acids. It is unclear at this moment the origin of these frame shifts, one possibility is that two different tssk 3 are present in mouse testis. More likely, one of these two sequences could have a small mistake in the sequence. Since the tssk 3 b human homologue was obtained independently from the mouse cDNA clone and has 100% homology at the amino acid level with its mouse homologue, applicants are confident about the accuracy of both the mouse 3b and the human tssk 3 sequences.

Chromosomal Mapping of Human and Murine Tssk3.

The chromosomal location of tssk 3b has also been mapped by fluorescence in situ hybridization (FISH) using the full length human cDNA probe. Fluorescent signals were detected on chromosome 1 in all 20 metaphase spreads scored. Among a total of 109 signals observed, 49 ( 45% ) were on 1 p. All chromosome-specific signals were localized to 1 p34.1-34.3. The distribution of signals was as follows: 1 chromatid (6 cells), two chromatids (14 cells) and three chromatids (5 cells). The mouse tssk 3b cDNA homologue mapped to the syntenic region in chromosome 4, band E.

### EXAMPLE 2

Cloning of the Full Length cDNA of the Human Homologues of the Tssk Kinase Family and a Partial cDNA Sequence from the Tssk Substrate.

Using the predicted mouse tssk 1 and 2 amino acid sequences for the purposes of searching the gene bank, a genome sequence of both mouse tssk 1 and tssk2 was obtained. These genes mapped to chromosome 5 and 22 respectively and are intronless. The sequences corresponding to the 3′ end and 5′ end of the coding region were used to design sense and antisense primers respectively. The antisense primers were used in 5′ RACE and the sense primer in 3′ RACE using human testis marathon ready cDNA (Clontech, Inc.) as the template in order to ultimately obtain a full length sequence of both human kinase homologs. The amplified cDNA fragments were cloned using the TOPO TA cloning kit (Invitrogen) and sequenced. To amplify the full length human tssk kinases cDNA, the 5′ end of the cDNA sequences obtained following 5′ RACE were used to design a 5′ sense primer. The 3′ end of the cDNA sequences obtained following 3′ RACE were used to design antisense primers. These two pairs of primers were then used to amplify human tssk1 and 2 from the same library. The amplified sequences 1.3 kb (tssk 1) and of 1.2 kb (tssk 2) were subcloned and sequenced. The translated human homologues of the three members of the tssk kinase family are provided as SEQ ID NOS: 4-6, respectively.

To analyze the specificity of expression, commercial blots depicting several human tissues were performed using random primed-labeled probes from the full length cDNA of the human tssk 1 and 2. In addition a random prime-labeled probe from a partial 800 base pair sequence was used to determine the tissue distribution of the tssk substrate. Northern blots were performed using a commercially available human tissue blot (Clonetech).

Northern blots reveal that tssk 1, 2 and tssk substrate are testis specific mRNAs. These same blots were stripped to the tssk probes and reprobed with a beta actin sequence, confirming that each of the lanes was equally loaded with RNA. To further analyze the specificity of expression, the same probes were used to perform dot blots using commercially available mRNA arrays from 76 different human tissues (using the Multiple Tissue Expression (MTE™) Array from Clonetech, Cat # 7775-1. The only signal obtained in each of the probed MTEs is in the grid containing testis RNAs. This experiment confirmed that these messages are testis specific in human. In addition, Kueng et al., (1997) demonstrated that tssk 1 and 2 are postmeiotically expressed in mouse germ cells and that these messages are not present in other 11 tissues.

### EXAMPLE 3

Immunolocalization and Immunoblotting Experiments

In order to determine if the tssk 1, 2 and 3 kinases and their substrate are present in the testis and/or mature sperm specific antibodies against the recombinant proteins will be generated. Alternatively, anti-peptide antibodies against specific peptides designed from the predicted amino acid sequence of each cDNA will be made. The specificity of each antibody generated will be tested against the recombinant tssk 1, 2 and 3. It is expected that the anti-peptide antibodies designed against specific amino acid sequences of each protein will be specific.

Antibodies made against tssk kinases and against the tssk substrate will be used to analyze for the presence of these kinases in other tissues. For this purpose, Clontech protein Medleys™ of different tissues such as brain, heart, kidney, liver, lung, ovary, placenta, skeletal muscle and spleen will be tested by western blot using the anti tssk and anti tssk substrate antibodies. Since mRNAs coding for tssk 1, 2 and 3 are only present in the testis, a similar protein distribution is expected. Since human homologues of tssk kinases have more than 80% homology when compared with their mouse counterparts, it is expected that antisera against the human recombinant tssk kinases recognize the mouse homologues as well. Thus, the antibodies made against the human tssk kinases will be also tested in mouse tissues.

Rats and rabbits will be used for production of polyclonal antibodies against the purified recombinant protein. Protocol as described by Mandal et al. (Biol. Reprod. 61 (1999), pp.1184-1197) will be followed for this purpose. Antibody titers will be monitored by ELISA and specificity of the antibody will be checked by SDS-PAGE and Western blotting analysis.

Sperm Preparation.

In order to perform immunolocalization and immunoblotting experiments human sperm will be collected from healthy donors and purified using Percoll (Pharmacia Biotech, Upsala, Sweden) density gradient centrifugation as previously described (Naaby-Hansen et al., Biol Reprod 1997; 56:771-787). Sperm will be then resuspended to a final concentration of 2×10⁷ cells/ml.

SDS-PAGE and Immunoblotting.

Sperm and other cell types from different tissues will be pelleted by centrifugation, washed in 1 ml of phosphate buffered saline (PBS), resuspended in sample buffer (Laemmli, 1970) without mercaptoethanol and boiled for 5 min. After centrifuging, the supernatant will be saved, 2-mercaptoethanol will be added to a final concentration of 5%, boiled for 5 min., and then subjected to 10% SDS-PAGE. Protein concentration will be determined by ABC kit from Pierce. Electrophoretic transfer of proteins to Immobilon P and immunodetection will be carried out as previously described (Kalab et al., Mol Reprod Dev. May 1994;38(1):91-3). Gels will be stained either with silver, coomasie blue or will be transferred to immobilon PVDF (Millipore) and probed with the anti recombinant antibodies.

Immunofluorescence.

To determine the intracellular location of tssk 1, 2 and 3, the specific antibodies against the soluble enzyme will be used in immunofluorescence experiments and immunoelectromicroscopy of human testicular tissue and human sperm. In addition, if the antibodies recognize the mouse antigens, the localization will be explored by immunofluorescence in mouse germ cells and sperm.

Sperm will be treated in the appropriate experimental conditions, fixed in suspension with a solution of 3% (w/v) paraformaldehyde-0.05% (v/v) glutaraldehyde in PBS for 1 h, washed in PBS at 37° C., and then permeabilized with 0.1% (v/v) Triton X-100 in PBS at 37° C. for 10 min. The sperm will then be washed in PBS and incubated overnight with serial dilutions (5, 10, 50 and 100) of the appropriate antibody as previously described (Visconti et al., 1996). After washing the sperm with PBS, they will be incubated with FITC-coupled goat anti mouse IgG and then attached to poly-lysine-coated microscope slides. Following 3×washes with PBS, the slides will be mounted with fluoromont and fluorescence will be assessed. Testicular samples obtained from testicular biopsies will be processed as previously described (Westbrook et al., Biol Reprod. August 2000;63(2):469-81).

### EXAMPLE 4

Expression of Recombinant Tssk Protein

Since many kinases have been expressed as active molecule in E coli (Bodenbach et al., 1994; Letwin et al., 1992). Advantage will be taken of E coli's relatively simple, easy- to scale-up. Open reading frames of tssk1, 2, 3 will be amplified and fused with a his tag in pET28b vector (Novagen). The plasmid will be transformed into BL21 DE3 or other appropriate host strain. Recombinant protein production will be induced by addition of IPTG to 1 mM final in the cultural medium. Recombinant protein will be purified from E coli lysate using Ni-NTA column under native condition. To facilitate crystal formation, highly purified protein is optimal. To purify the protein preparation from above, preparative electrophoresis using PrepCell (BIO-RAD) will be employed. Kinase assay will be conducted before proceeding to crystallography.

To produce recombinant testicular tssk kinases and tssk substrate in bacteria, expression constructs of tssk 1, 2 and 3 as well as for the tssk substrate will be made. The coding region of each of these proteins will be subcloned in the pET28b expression vector that contains at the C-terminus 6 residues of His-tag. In particular, the constructs will be made using complete ORF primers that are designed to create an NdeI site at the 5′ end and an XhoI site at the 3′ end. The amplified products will be ligated into the NdeI-XhoI sites of pET28b expression vector. Since the recombinant protein will be fused with the 6 histidine residues of the expression vector the expressed protein will be purified using Ni-Histidine bind resin affinity chromatography. Once purified, kinase activity will be evaluated with the tssk substrate to make sure that the enzyme has folded correctly.

Tssk 2 protein was successfully expressed in E. coli and purified. To express tssk 2, the open reading frame was subcloned into a pET28b expression vector carrying a His-tag. Recombinant protein was produced following induction with IPTG. Bacterially expressed and partially purified tssk2 was further purified by preparative gel electrophoresis using a PrepCell from Biorad. The fractions were collected and analyzed by SDS-PAGE. Tssk substrate was expressed and purified similarly. These purified proteins were used to produce rat polyclonal antibodies using standard techniques.

Since multiple kinases have the property to become autophosphorylated in vivo and in vitro, purified bacterially expressed recombinant tssk 2 was assayed for autophosphorylation. The experiment was conducted in the presence of 40 μM ATP (1 μCi of [³²p] ATP), 10 mM Mg²⁺, phosphatases inhibitors such as p-nitro-phenyl phosphate and glycerol phosphate and proteases inhibitors (leupeptin and aprotinin 10 μg/ml), the assay was stopped with sample buffer and tssk 2 was separated in 10% PAGE. Auto radiography of the dried gel showed that recombinant tssk 2 incorporated ³²p, suggesting that the bacterially expressed tssk 2 folded correctly for phosphorylation to occur and could be used for structural studies.

Since not all bacterial expressed recombinant proteins are folded correctly, a similar approach will be taken to express tssk 1, 2 and 3 in yeasts. In order to express the tssk kinases and substrate in yeasts, the kinases will be subcloned in pPICZαB vector from Invitrogen (Carlsbad, Calif.) and will be expressed in Pichia pastoris as secreted protein as well as an intracellular protein. These constructs also have a C-terminal His tag that will allow an easy purification of the recombinant proteins either from the culture media or from the extracted cells.

### EXAMPLE 5

Tssk 2 Interacts with Tsks in a Yeast Two-Hybrid System

To demonstrate protein-protein interaction between tssk 2 and tsks a two yeast hybrid system was utilized. In this experiment, a bait gene (tssk 2) was first transformed into the reporter strain as a fusion to the GAL4 DNA binding domain (DNA-BD). A second plasmid that expressed tsks as fusion to the GAL 4 activation domain (AD) was also introduced into the AH109 reporter strain. Western blots were performed to confirm expression of fusion proteins in yeast. Interaction between tssk 2 and tsks was observed to promote transcription of the Histidine (HIS) gene and allow for the growth of yeast in His-free medium. Similarly, cotransfection of yeast cells with a first construct expressing a p53 fused to the GAL4 DNA binding domain and a second construct expressing the SV40 Large T antigen fused to the GAL 4 activation domain (AD) demonstrated that p53 and the SV40 Large T antigen interacted and promoted His gene activation. This interaction was used as a positive control. In contrast, fusion proteins of tssk 2 and p53 with the DNA-BD did not promote growing when cotransfected with the GAL-AD plasmid. Neither GAL-AD alone nor the fusion proteins between GAL-AD and TSKS or GAL-AD and SV40 Large T antigen had the ability to activate transcription of the His gene. This experiment demonstrated that tssk 2 and the substrate tsks are able to interact, suggesting that the human homologues of these proteins behave in a similar way to their mouse counterparts.

To investigate whether tssk 2 and tsks also interact in vivo, capacitated and non capacitated sperm will be extracted with Triton×100 in conditions that protect protein-protein interaction and then immunoprecipitation will be conducted with specific antibodies. The interaction will be assayed by Western blots with the other antibody in a typical cross immunoprecipitation experiment. Since proteins that interact should co-localized, double labeling immunofluorescence will be conducted and co-localization investigated. Although rabbit antibodies have not yet been obtained for the human tssks, rat anti tssk 2 and rat anti tsks antibodies have been previosly obtained and therefore there is no reason to anticipate any difficulties in obtaining antibodies to the human tssks.

### EXAMPLE 6

Isolation of the Crystal Structure of Tssk 1, 2, 3 and the Tssk Substrate

Two strategies will be followed for the rationale design of tssk-specific inhibitors. First, an in vitro kinase assay compatible with High Throughput screening will be developed. Second, crystal structure of tssk kinases alone or complexed with their substrate will be obtained.

For structural studies using X-ray crystallography, it is necessary to obtain approximately 5 mg of the active kinases. This amount of protein is generally a suitable quantity for initial crystallization trials. Initial trials will be performed on the recombinant intact protein; this is a common crystallization technique that has proven to be successful in many cases, including several structural studies of enzymes. Prior to crystallization screening, the expressed fragment would be checked for a suitable level of enzymatic activity as well as for purity, homogeneity and solubility. Several commercially available crystallization screening kits, used in conjunction with the hanging drop vapor diffusion method, provide the standard first step in the search for crystal growth conditions. Crystallization screening of the fragment in the presence of catalytically required metal ions, substrates and/or inhibitors will be carried out simultaneously with the screen of the apoenzyme.

Crystals will be obtained at 21° C. by equilibrating sitting drops. For cryodiffraction experiments, the crystals will be transferred to a similarly buffered solution through three solutions with intermediate concentrations of these reagents. For diffraction experiments at room temperature and lower resolution, Cu Kα radiation from rotating anode X-ray generators will be used. Final structure will be established after preliminary refinement, model improvement and further improvement.

In the case of the tssk kinase family, crystallization in the presence of their substrate will be attempted in order to establish a molecular basis of the kinase activity. Once initial crystallization conditions are obtained, they will be optimized in order to obtain crystals that diffract to at least 3.0 Å resolution. Direct phasing of the structure via the MAD (multiple wavelength anomalous dispersion) and MIR (multiple isomorphous replacement) techniques would be carried out simultaneously, to assure success. Semi-automated model-building and refinement techniques for phased structures would be utilized to achieve rapid structural results. Once a structure is obtained, the results will be analyzed with a view to understanding the unique role of the tssk kinase family in spermatogenesis and/or sperm function. An ultimate goal of these studies would be the use of the structure as a template for the design of specific inhibitors of tssk 1, 2 and/or 3, which may prove useful as contraceptives.

### EXAMPLE 7

Development of a Tssk-Specific Kinase Assay.

The design of a specific kinase assay for the tssk family is advantageous from different points of view. First, a kinase assay will allow for the characterization of the kinetic properties of these enzymes such as the Km for ATP, divalent cation and substrate. Second, since only active enzymes are suitable for crystallographic studies, a kinase assay will confirm the folding of the recombinant proteins. Third, it is desirable to develop an in vitro kinase assay at the lab scale to be the basis to a kinase assay appropriate for high throughput screening of tssk-specific inhibitors.

Development of assays to measure tssk kinase activity will follow general characteristics of kinase assays such as presence of substrate, [γ32P]ATP, Mg²⁺ and/or Mn²⁺ and phosphatases inhibitors. First however a source of tssk kinase must be provided. The tssk kinase will be generated by recombinantly expressing these proteins and purifying the expressed kinase.

Kinase Assay in Mammalian Cell Extracts.

ORFs of the respective human tssk kinases will be subcloned in a pCMV-HA epitope-tagged mammalian expression vector (Clontech cat # K6003-1). Similarly, the ORF of the tssk substrate will be subcloned in a pCMV-Myc mammalian expression vector (Clontech, same as above). Each of the three HA-kinases will be coexpressed with the cMyc-tssk substrate in three separate COS cell lines for each of the respective human tssks. Coexpression will be validated performing immunofluorescence with the respective antitag antibodies. Antibodies against HA and Myc are available from Clontech. Since anti c-Myc is a mouse monoclonal and anti HA is a rabbit polyclonal, it will be possible to analyze whether both the kinase and the substrate were coexpressed in the same cells.

Proteins will be then extracted with 1% Triton and immunoprecipitation will be performed using anti HA-tag antibodies. Alternatively, anti tssk-kinase/anti-tssk substrate antibodies could be used to immunoprecipitate the tssk kinases and tssk substrate. Typically the antibodies will be linked to a solid support such as a sepharose bead to assist in the isolation of the target ligand. After precipitation with Protein A sepharose, the pellet will be assayed for kinase activity using different concentrations of ATP (10 μM, 100 μM and 1 mM), Mg²⁺ or Mn²⁺ (100 μM, 1 mM and 10 mM) and 1 μCi of [γ32P] ATP. The phosphorylated protein will be evaluated following SDS-PAGE separation and autoradiography. The evaluation of kinase activity after immunoprecipitation is a frequently used method that allow for specific measurement of the activity of one particular kinase (Coso et al., 1995, Cell 81, 1137-46; Moos et al., 1995, Biol Reprod 53, 692-9). Since Kueng et al. (1997) has successfully detected the phosphorylation of tssk substrate after immunoprecipitation of tssk 1 and 2, it is expected that it will be possible to measure kinase activity of tssk kinases after coexpression in a mammalian system.

In vitro Kinase Assay.

Each purified enzyme will be mixed with different concentrations of ATP (10 μM, 100 μM and 1 mM), Mg²⁺ or Mn²⁺ (100 μM, 1 mM and 10 mM), 1 μCi of [γ32P] ATP and different concentrations of the purified tssk substrate (1, 10 and 100 ng/assay). Phosphorylation will be evaluated following SDS-PAGE separation and autoradiography. It is expected that if the proteins are correctly folded the phosphorylation of the substrate will be easily detected with this methodology.

### EXAMPLE 8

Generation of Tssk Antibodies

Purified tssk 2 and tsks were used to produce rat polyclonal antibodies. Antisera against recombinant human tssk 1, 2 and 3 as well as to the human tssk substrate will be employed to define their tissue distribution and subcellular localization at the protein level. The rat anti-tssk and anti-tsks antibodies recognized the recombinant protein and also proteins in sperm and testes with the predicted MW, suggesting that at least one member of the tssk family and their substrate (tsks) are present in sperm. These antibodies were then used to study the intracellular localization of these proteins in capacitated human sperm. Tssk 2 was observed to localized to the equatorial segment of human sperm. Tsks also localized to the equatorial segment. Nevertheless anti-tsks antibody also recognize proteins present in the anterior head and in the tail. Immunolocalization of these molecules suggests that tssk 2 and tsks are present in similar region of the sperm at least in a fraction of the human sperm population. Control experiments were performed using rat pre immune serum of the respective antibody. Both Western blots and immunofluorescence were negative. Although the antibody against tssk 2 was produced against tssk 2, we can not discard that this antibody recognized other members of the tssk family since their sequences have high homology. However, discrimination between the three tssk isoenzymes, may be possible by generating antibodies against the C-terminal domain that is different between tssk 1 and 2 and is not present in tssk 3.

### EXAMPLE 9

Real-Time RT-PCR Analysis.

First strand cDNA was prepared from 5 μg of total RNA from human testis (Ambion, Austin, Tex.) using 1 μM oligo d(T) primer (Ambion, Austin, Tex.) and Omniscript reverse transcriptase (Qiagen, Alameda, Calif.) in a 100 μl reaction using the reaction buffer supplied by the manufacturer. Real-time RT-PCR was performed using a Bio-Rad (Hercules, Calif.) i-cycler IQ system. Primer pairs were validated in 20 μl PCR reactions containing 2 μl of testis cDNA, 10 μl of IQ SYBR Green supermix (BioRad), and 0.6 μl of a 10 μM stock of each primer (0.3 μM final concentration). Cycle conditions were 95° C. for 3 minutes followed by 45 cycles of 95° C. for 10 seconds and 60° C. for 30 seconds. The amplification was followed by melting curve analysis in which the PCR products were denatured at 95° C. for 1 minute and annealed at 72° C. for 10 seconds. The temperature was then increased in 0.1° increments while monitoring the loss of SYBR green fluorescence. Each primer pair amplified a single product with a sharp melting peak at a temperature consistent with the calculated Tm of the predicted PCR product. Agarose gel electrophoresis confirmed that the amplified products had the expected sizes. No products were obtained when reverse transcriptase was omitted from the cDNA synthesis reactions.

The oligonucleotide primers were all purchased from Qiagen Operon (Alameda, Calif.). Where possible, the primers were selected to span a splice junction. Obviously, this could not be done in cases of intronless genes such as TSSK 1 and TSSK 2. Primer pairs for each gene were: TSSK 1, GCCCCTAGGTGGATGAGG (forward; SEQ ID NO: 21) and TCACGCTCTGGGGGAGTA (reverse; SEQ ID NO: 22); for TSSK 2, GGGTTCCTACGCAAAAGTCA (forward; SEQ ID NO: 23) and GTTTTCTTGCGGTCGATGAT (reverse; SEQ ID NO: 24); for TSSK 3, GGGGAAGGGACCTACTCAAA (forward; SEQ ID NO: 25) and GTCCAGGGTACGGACGATTT (reverse; SEQ ID NO: 26); for TSSK 4, TACGCGTCACCCGAGTGCT (forward; SEQ ID NO: 27) and ACGCCCATGCTCCACACA (reverse; SEQ ID NO: 28); for TSKS, GCTGAGCGAGAATCTGGAG (SEQ ID NO: 29) and TTCAGCATCTTCCACAGACC; (SEQ ID NO: 30); for TSKS-1 GGATTCAAATGAGGCTCCAAC (forward; SEQ ID NO: 31) and TGGAGGTAGCGCAGCTTCT (reverse; SEQ ID NO: 32); for glyceraldehyde-3-phosphate dehydrogenase (GAPDH), ATCATCAGCAATGCCTCCTG (forward; SEQ ID NO: 33) and ATGGCATGGACTGTGGTCAT (reverse; SEQ ID NO: 34).

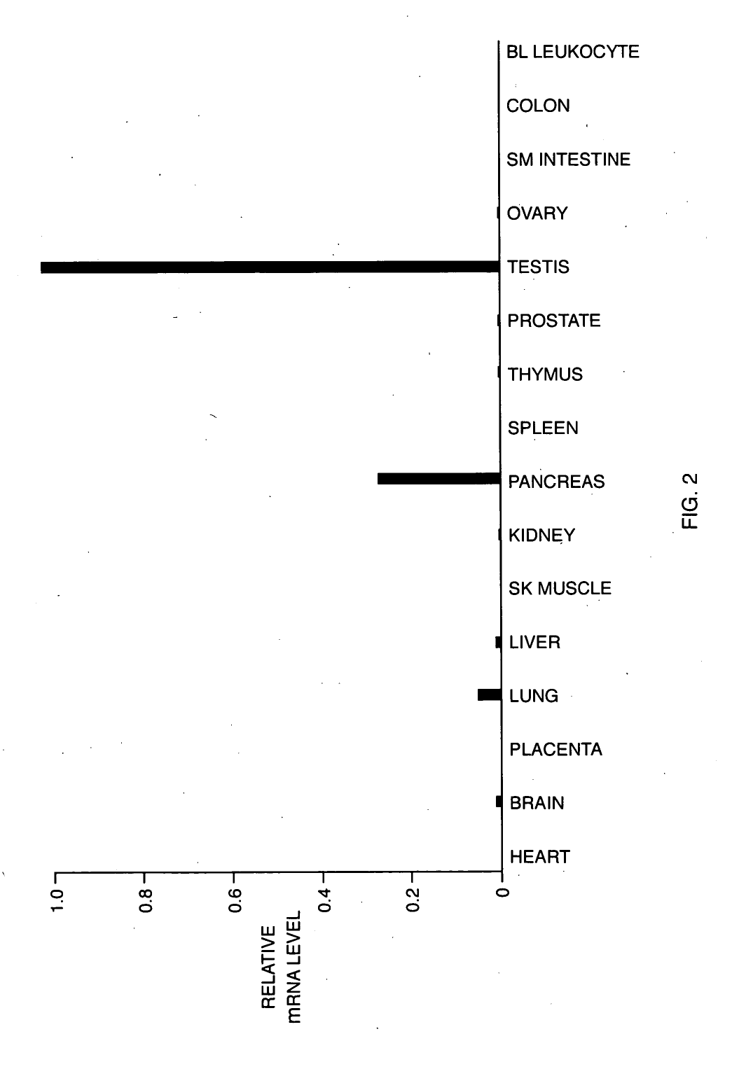

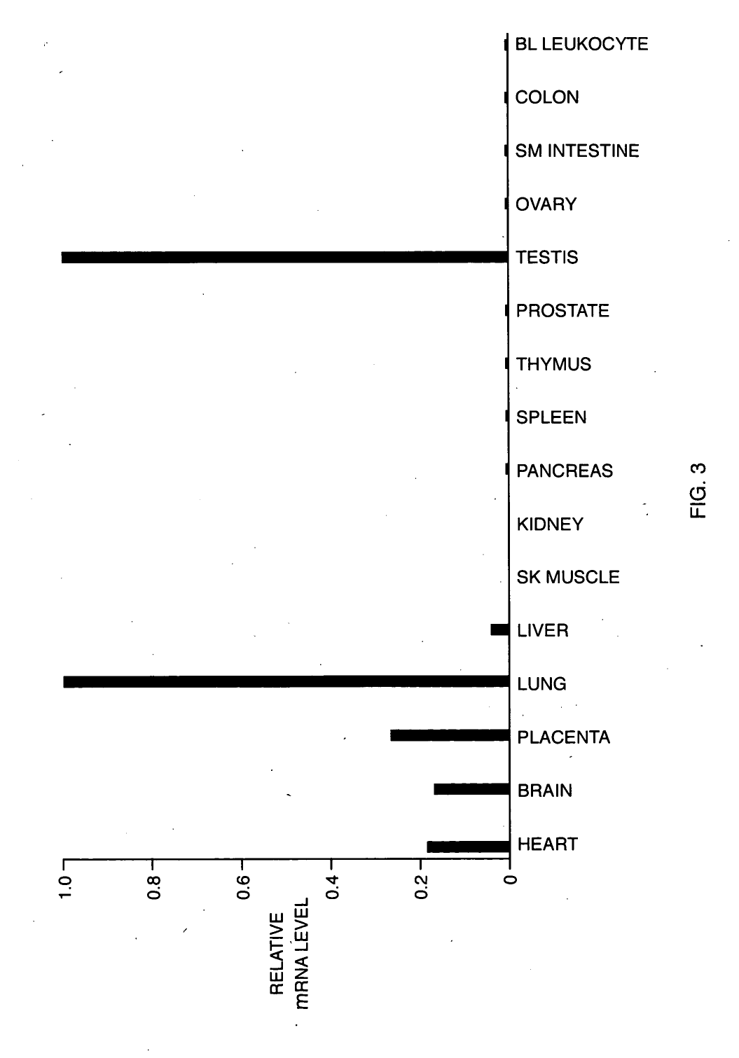

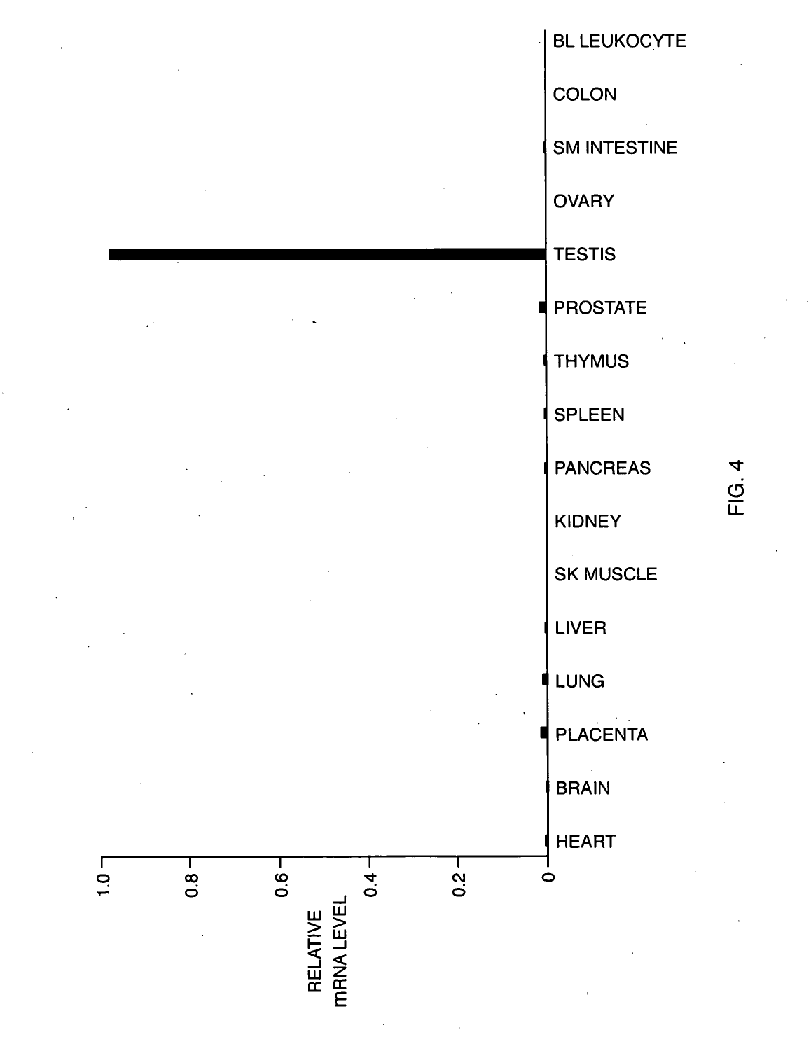

Relative levels of mRNA expression in human tissues were determined using multiple tissue cDNA (MTC) panels from BD Biosciences (Palo Alto, Calif.) in real-time PCR reactions as described above. The point at which the PCR product is first detected above a fixed threshold, termed cycle threshold (Ct), was determined for each sample. Melting curve analysis confirmed the amplification of only the expected product. To determine the quantity of gene-specific transcripts present in each tissue cDNA relative to testis, their respective Ct values were first normalized by subtracting the Ct value obtained from the GAPDH control (e.g. —ΔCt=Ct TSSK 3−Ct GAPDH). The concentration of gene-specific mRNA in a given tissue relative to testis was then calculated by subtracting the normalized Ct values obtained with each tissue from that obtained with testis (e.g. —ΔΔCt=ΔCt of brain−ΔCt of testis), and the relative concentration was determined (relative concentration 32 2^{-ΔΔCt}). See FIGS. 2-5.

### EXAMPLE 10

In Situ Localization and Northern Blot Analysis of Tssk4

In Situ Analysis

The probe used for in situ hybridizations was prepared from the Mus musculus serine/threonine protein kinase SSTK (Sstk-pending), mRNA. NM 032004 (SEQ ID NO: 35). The amplified region is a 394 base pair region of the coding sequence ranging from 320-714 of (SEQ ID NO: 36). The forward primer had the sequence tcg agt tca tcg aag tgt gc (SEQ ID NO: 37) and the reverse primer had the sequence ggt gac cat gac gta gag ca (SEQ ID NO: 38).

Materials:

The template comprised a mouse testis cDNA first strand kit (Invitrogen) and the components used in the PCR reaction and their concentrations are as follows:

| components | final concentration | 1 × 50 μl |
|---|---|---|
| Aqua bidest | | 40 |
| 10 × PCR Reaction buffer | 1x | 5 |
| NTP's, 10 mM | 0.2 mM of each | 1 |
| MgCl₂, 50 mM | 1.5 mM | 1.5 |
| Tag polymerase | 2.5 units | 0.5 |
| Primer out of stocksolution | | 1 μl for + 1 μl rev |
| 1:10 diluted | | |
| Template (cDNA mouse | | 1 μl |
| testis) | | |

| | | |
|---|---|---|
| 1 | 94° C. | 1 min |
| 2 | 92° C. | 30 sec |
| 3 | 66° C. | 30 sec |
| 4 | 72° C. | 2 min |
| 5 | Go to 2, 33x | |
| 6 | 72° C. | 10 min |
| 7 | 4° C. | |

Agarose gel analysis confirmed the amplification of the 394 fragment, which was then cloned into a pCR 4 TOPO vector using the TOPO TA Cloning Kit for sequencing (Invitrogen No.: K4575-01). The resulting plasmids were isolated using Qiagen Qiaprep Spin (Qiagen No: 27106). The mouse TSSK 4 sequence in the clone to be used for the in situ hybridization was verified by sequencing the insert within in the plasmid.

The plasmid was linearized with either Not I (antisense) or Pme I (sense). Labeled sense and antisense cRNA were synthesized by incubating a linearized template either in presence of T3- RNA polymerase (antisense probe, NotI linearized) or with T7- RNA Polymerase (sense probe, Pme I linearized). Labeling was performed using the method outlined in the digoxygenin (DIG)-RNA-labeling kit from Boehringer (Mannheim, Germany).

In Situ Hybridization

6 hours Bouin's-fixed and paraffin-embedded mouse testis were sectioned at 4 μm. After deparaffinization in Pro Taqs Clear Intermedium (Quartett GmbH, Berlin, Germany) and rehydration through a series of ethanol gradient solutions, tissues were incubated for 20 minutes in 0.2N HCl at room temperature and then 15 minutes at 70° C. in 2×SSC. The slides were washed in PBS at room temperature for 5 min. This was followed by a partial digestion with proteinase K (2 μg/ml final) for 10 minutes at 37° C. After incubation in 0.2% (w/v) glycine for 10 minutes at 4° C. and postfixing with 4% paraformaldehyde (in PBS) for 5 minutes at 4° C., the sections were washed two times in PBS at room temperature.

The sections were acetylated in 0.1 M ethanolamin and 0.25% (v/v) acetic anhydride for 5 minutes at room temperature and after that washed two times in PBS at room temperature. This was followed by the prehybridization step for at least 3 hours at room temperature. The prehybridization solution contained 50% (w/v) formamide, 5×SSC, 5× Denhardts (Denhardts: 1% w/v) bovine serum albumin, 1% (w/v) ficoll, 1% (w/v) polyvinylpyrolidone in DEPC water), tRNA 400 μg/ml final (bakers yeast transfer RNA, Roche) and hering sperm DNA 200 μg/ml final (Gibco). Hybridization buffer consisted of prehybridization buffer and 20-50 ng of DIG-labeled sense or antisense probes. Hybridization was performed at 55° C. overnight. Excess probe was removed by sequential washing steps for 5 minutes with 2×SSC at room temperature. After incubation for 30 minutes with 2 mg/ml RNAse A in 10 mM Tris/HCl (pH 8.0), 0.5 M NaCl, 5 mM EDTA (pH 8.0) at 37° C., 3 washes were performed. (At first with 2×SSC at 37° C. for 5 minutes, then with 1×SSC at 37-55° C. for 15 minutes, after that with 0.2×SSC at 55° C. for 15 minutes). At the end the sections were washed in 1×TBS for 5 minutes at room temperature.

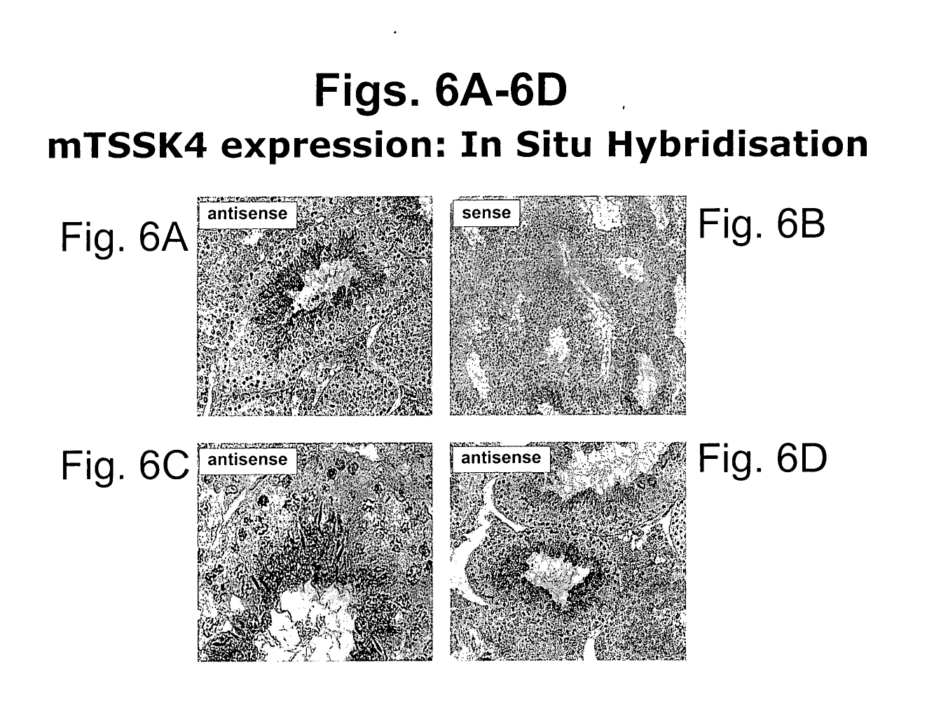

The hybridized probe was localized on the sections after incubation with anti-DIG alkaline phosphatase (Boehringer) for 1 hour, followed by 3 washing steps in maleineacid buffer each for 10 minutes, and visualized by addition of substrates (nitroblue-tetrazolium chloride and 5-bromo-4-chloro-3-indolyl phosphate solution [NBT/BCIP]). Incubation was performed in the dark overnight at room temperature. The sections were then counterstained with Mayer's hematoxylin and mounted in GVA mounting solution (Zymed Laboratories, San Francisco, USA). The results are shown in FIG. 6.

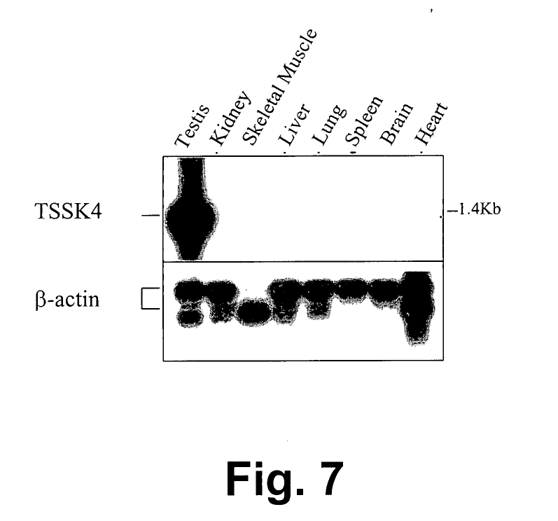

A Northern blot membrane containing 1 microgram of mouse poly-A mRNA in each lane (BD Biosciences) was hybridized to a 1.4 kilobase mouse tssk 4 cDNA probe labeled with ³²P-dCTP by random priming. Prehybridization was performed at 65° C. for 2 hours first in ExpressHyb purchased from BD biosciences without probe. Hybridization was carried out successively by replacing the ExpressHyb with a new batch after adding the labeled probe at the same temperature overnight. The blot was washed with 2×SSC, 0.1% SDS twice at room temperature and twice with 0.1 ×SSC, 0.1%SDS at 60° C. The signal was detected by exposing the blot to an x-ray film at −80° C. for overnight. Hybridization to beta-actin control was performed under the same condition as above after striping off the radioactive probe by boiling the Northern membrane. The film was exposed for 6 hours. Results are shown in FIG. 7, wherein the only detectable signal appears in testis mRNA.

1. A method for identifying antagonists of tssk kinase activity, said method comprising
providing an in vitro kinase assay composition, said composition comprising a labeled source of phosphate, a tssk substrate and a tssk 4 kinase;
contacting said composition with a candidate inhibitory compound to form a test assay composition;
incubating the test assay composition under conditions permissive for kinase activity in the absence of a kinase inhibitor; and
identifying those compounds that decrease the activity of the tssk kinase.

2. The method of claim 1 wherein the labeled source of phosphate is [³²p] Atp:

3. The method of claim 2 wherein the tssk substrate comprises an amino acid sequence of SEQ ID NO: 8.

4. The method of claim 1 wherein said kinase assay composition further comprises tssk kinases selected from the group consisting of tssk 1, tssk 2 and tssk 3.

5. The method of claim 1 further comprising the step of conducting a second reaction wherein an in vitro kinase assay composition, comprising a labeled source of phosphate, a tssk substrate and a tssk 4 kinase, is incubated under conditions permissive for kinase activity, and comparing the amount of labeled tssk substrate produced to the amount of labeled tssk substrate produced by the test assay composition.

6. The method of claim 1 further comprising the step of conducting a second reaction wherein the candidate compound is contacted with a control composition wherein the control composition comprises the candidate compound, a labeled source of phosphate, a kinase substrate and a non-tssk kinase, and determining if the candidate compound decreases the activity of the non-tssk kinase.

7. A method of decreasing the fertility of a male mammalian species, said method comprising the steps of administering a composition comprising an inhibitor of tssk 4 kinase activity.

8. A recombinant human tssk gene construct, said construct comprising a non-native promoter operably linked to the nucleic acid sequence of SEQ ID NO: 19.

9. A transgenic cell comprising the construct of claim 8.

10. An antibody that binds to the polypeptide of SEQ ID NO: 20.

11. The antibody of claim 10 wherein the antibody does not bind to the peptide sequences of SEQ ID NOs: 4-6.

12. The antibody of claim 10 wherein said antibody is a monoclonal antibody.

13. A method of screening for potential human therapeutic agents, said method comprising contacting a tssk polypeptide selected from the group consisting of SEQ ID NO: 20 and bioactive fragments of SEQ ID NO: 20 with a candidate compound under physiological conditions;
washing the tssk polypeptide to remove unbound and non-specific bound material; and isolating compounds that remain bound to the tssk polypeptide.

14. The method of claim 13 wherein the tssk polypeptide is immobilized on a solid surface and the candidate is labeled.

15. The method of claim 13 further comprising the step of determining if the candidate compound selectively binds to said tssk polypeptide relative to other kinase polypeptides.
